(19) United States
(12) Patent Application Publication
(10) Pub. No.: US 2022/0403343 A1
(43) Pub. Date: Dec. 22, 2022

(54) METHODS OF REPROGRAMMING A CELL

(71) Applicants: THE UNIVERSITY OF WESTERN AUSTRALIA, Crawley, Western Australia (AU); MONASH UNIVERSITY, Clayton, Victoria (AU)

(72) Inventors: Ryan LISTER, Crawley, Western Australia (AU); Sam BUCKBERRY, Crawley, Western Australia (AU); Jose POLO, Clayton, Victoria (AU); Xiaodong LIU, Clayton, Victoria (AU)

(21) Appl. No.: 17/779,275

(22) PCT Filed: Nov. 26, 2019
(86) PCT No.: PCT/AU2019/051296

§ 371 Date: May 24, 2022

(51) Int. Cl. C12N 5/074

(52) U.S. Cl. CPC C12N 5/0696; C12N 2501/60; C12N 2506/1307; C12N 2506/45

(57) ABSTRACT

The present invention relates to methods for producing an induced pluripotent stem (iPSC), the method comprising the following steps in order: culturing a somatic cell in a first culture condition adapted to promote the reprogramming of the cell towards a pluripotent state; culturing the cell in a second culture condition adapted to promote a hypomethylated DNA state in the cell; and culturing the cell in a third culture condition adapted to promote a primed pluripotent state, thereby producing an iPSC from a somatic cell. The invention also relates to cells and compositions resulting from those methods.

### FIELD OF THE INVENTION

The present invention relates to improved methods for reprogramming a somatic cell to a pluripotent state, and cells and compositions resulting from those methods.

### BACKGROUND OF THE INVENTION

The reprogramming of somatic cells into pluripotent cells that closely resemble human embryonic stem cells (ESCs) is an area to technology that holds great potential in biomedical research and regenerative medicine.

Induced pluripotent stem cell (iPSC) technology typically involves the genetic manipulation of somatic cells or introduction of defined factors to somatic cells, in order to generate pluripotent cells. The iPSCs produced by these methods can then be used to generate any number of different cell types, through the application of specific differentiation conditions to the iPSCs.

Cell reprogramming requires resetting the somatic epigenome to closely resemble the epigenome of an ESC. However, multiple studies have revealed that using current reprogramming approaches, iPSCs typically retain epigenetic patterns, such as DNA methylation, of the original somatic cells, and are therefore epigenetically distinct from ESCs. In other words, the iPSCs possess an epigenetic memory of the somatic cell from which they are derived.

In addition, iPSC-specific epigenetic abnormalities frequently arise during the reprogramming process. These are abnormal/aberrant epigenetic (e.g. DNA methylation) patterns and states that are not present in the cell of origin or in ESCs. These aberrant DNA methylation patterns are stable through extensive iPSC passaging, can be transmitted through iPSC differentiation and are linked to inappropriate transcriptional activity in derived differentiated cells.

The epigenetic memory of the somatic cell and aberrant epigenetic (e.g., DNA methylation) states impact functionally on the resulting iPSCs, altering transcription and biasing iPSC differentiation potential towards the original somatic cell lineage. Thus, iPSCs harbour epigenome states and epigenome memory that can affect differentiation and function. Further, incomplete epigenetic reprogramming may be a significant limitation for iPSC technology and therapeutic application.

Accordingly, there is a need for improved methods for reprogramming of somatic cells towards a pluripotent state.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In a first aspect, the present invention provides a method of producing an induced pluripotent stem cell (iPSC), the method comprising the following steps in order:
(a) culturing a somatic cell in a first culture condition adapted to promote the reprogramming of the cell towards a pluripotent state;
(b) culturing the cell in a second culture condition adapted to promote a hypomethylated DNA state in the cell, wherein the culturing in the second culture condition is for a period of time that is insufficient to allow the cell to achieve an established naïve pluripotent state; and
(c) culturing the cell in a third culture condition adapted to promote a primed pluripotent state,

thereby producing an iPSC from a somatic cell.

In a second aspect, the present invention provides a method for reprogramming a somatic cell to an induced pluripotent cell (iPSC), the method comprising the following steps in order:
(a) increasing the protein expression or amount of one or more factors in the somatic cell, wherein the factors are for reprogramming the somatic cell towards a pluripotent state;
(b) culturing the cell in a first culture medium, for a sufficient time and under conditions to allow the reprogramming of the cell towards a pluripotent state;
(c) culturing the cell in a second medium adapted to induce a hypomethylated DNA state, for a sufficient time and under conditions to reset the epigenomic profile of the cell; and
(d) culturing the cell in a third culture medium adapted to induce a primed pluripotent state, for a sufficient time and under conditions to convert the cell to a primed pluripotent state;

thereby reprogramming the somatic cell to an iPSC.

In a further aspect, the present invention provides a method for reprogramming a somatic cell to an induced pluripotent stem cell (iPSC), the method comprising the following steps in order:
(a) increasing the protein expression or amount of one or more factors in the somatic cell, wherein the factors are for reprogramming the somatic cell towards a pluripotent state;
(b) culturing the cell in a first culture medium, for a sufficient time and under conditions to allow the reprogramming of the cell towards a pluripotent state;
(c) contacting the cell with a culture medium adapted to induce a hypomethylated DNA state and culturing the cell for a sufficient time and under conditions to allow reprogramming of the cell towards a hypomethylated DNA state;
(d) contacting the cell with a primed culture medium and culturing the cell for a sufficient time and under conditions to allow reprogramming of the cell to a primed pluripotent state;

thereby reprogramming the somatic cell to an iPSC.

The methods of the present invention are particularly suitable for minimising or reducing the likelihood of the generation of aberrant epigenetic patterns in the iPSCs. Accordingly, an iPSC obtained or obtainable by the methods of the present invention has an epigenomic profile that more closely resembles that of an embryonic stem cell (ESC) than an iPSC obtained or obtainable by conventional reprogramming methods.

As used herein, resetting the epigenomic profile of the cell refers to establishing an epigenetic profile that resembles the epigenetic profile of an ESC.

In any embodiment, a cell exhibiting a hypomethylated DNA state may be a naïve pluripotent cell.

The epigenomic profile of a cell obtained or obtainable according to the present invention can be compared to that of an ESC by determining a genomic methylation pattern for at least a portion of an iPSC obtained according to the present methods and comparing this to the methylation pattern of an ESC.

In any aspect of the invention, the iPSCs obtained or obtainable by the methods of the present invention are characterised by a non-CG DNA methylation profile that is similar to the non-CG DNA methylation profile of an ESC. In any aspect of the invention, the iPSCs obtained or obtainable by the recited methods, establish a H3K9me3 methylation state in the cell that resembles the H3K9me3 methylation state of an ESC. In any aspect of the invention, resetting the epigenetic profile refers to a level of transposable element DNA methylation and/or expression that is similar to the level of DNA methylation and/or expression of a transposable element in an ESC.

In any aspect of the invention, the iPSCs obtained or obtainable by the methods of the invention have a level of CG DNA methylation, and/or level of CG DNA methylation for a transposable element, that is similar to the level of methylation in an ESC at a locus as defined in any of Tables 3 or 4, herein.

In any aspect of the invention, the somatic cell is a human somatic cell and the iPSC produced according to the method is a human iPSC.

In certain embodiments of the invention, the period of time for culturing the cell to commence reprogramming towards a pluripotent state is at least 1 day following contacting the somatic cell with the first culture medium (according to the first aspect of the invention) or following increasing the protein expression, or amount of the one or more factors (according to the second or third aspects of the invention). The period of time may be 2, 3, 4, 5, 6, 7, or more days after contacting the cell with the first culture medium, or increasing the protein expression, or amount of the one or more factors. In any embodiment, the period of time for culturing the cell to commence reprogramming towards a pluripotent state may be any period of time provided that it enables the reduction of one or more markers and/or characteristics associated with the somatic cell.

The second culture medium or second culture condition, adapted to induce a hypomethylated DNA state, may be any culture condition or medium that promotes global DNA hypomethylation of the cell. In certain embodiments, the culture condition or medium may comprise any culture medium adapted for promoting a naïve pluripotent state or a state exhibiting characteristics of the naïve pluripotent state, such as global DNA hypomethylation. Examples of such media are known in the art and are further described herein. In certain preferred embodiments, the naïve medium comprises a MEK inhibitor, a PKC inhibitor, a GSK3 inhibitor, a STAT3 activator, and a ROCK inhibitor.

As used herein, global DNA hypomethylation refers to a reduction in the average level of DNA methylation across the genome of a cell, as compared to the level of methylation observed in a somatic (differentiated) cell type, or optionally a primed human ESC.

The primed medium (or third culture condition or third culture medium) may comprise any culture medium adapted for promoting a primed pluripotent state. Examples of such media are known in the art and are further described herein. In certain embodiments, the primed medium is selected from: Essential 8, KSR/FGF2, mTeSR, AKIT or B8.

In any aspect, the culturing of the cell in the second culture condition/culture medium may be for a period of time that is not sufficient for the development of pluripotency.

More specifically the timing of step c) in any aspect of the invention may include contacting the cell with media for inducing DNA hypomethylation or naïve medium for a period of time that is not sufficient to achieve a naïve pluripotent phenotype for the cell, but is sufficient to achieve global DNA hypomethylation in the cell. In other words, the timing of step d), i.e., contacting the cell with primed medium, may be performed before the cell has completed reprogramming and before the cell achieves a naïve pluripotent phenotype. In certain embodiments, the timing of step c) is once the cell has achieved global DNA hypomethylation but does not express one or more markers of naïve pluripotency markers, including markers of naïve pluripotency as described herein.

In alternative embodiments of the second and third aspects of the invention, the timing of step c) may be after the cells have become established naïve pluripotent cells including wherein the cells express one or more markers of naïve pluripotency as described herein.

As used herein, “naïve pluripotent state” or “naïve pluripotent phenotype” may also be understood to refer to a cell morphology or phenotype that includes cells that are round, dome-shaped. A naïve pluripotent state may also comprise a cell that has global DNA hypomethylation. A naïve pluripotent state may also comprise the expression of 1, 2, 3, 4, 5, 6, 7, 8, 9, 10, 11, 12, 13, 14, 15, 16, 17, 18, 19, 20, 21, 22, 23 or all markers selected from: KLF2, KLF4, TFCP2L1, TBX3, REX1, GBX2, STELLA (DPPA3), KLF17, DPPA5, TFCP2L1, MAEL, UTF1, ZFP57, DNMT3L, FGF4, FOXR1, ARGFX, TRIM60, DDX43, BRDT, ALPPL2, KHDC3L, KHDC1L and PRAP1 or other marker of naïve pluripotency as described herein, including in Table 5.

As used herein, a “primed pluripotent state” or “primed pluripotent phenotype” typically refers to a cell phenotype or morphology characterised by the presence of flat cell colonies. In certain embodiments, a primed pluripotent state refers to a pluripotent cell that expresses one or more mRNAs of post-implantation epiblast specific transcription factors. For example, primed cells may express 1, 2, 3, 4, 5, 6, 7, 8, 9, 10, 11, 12, 13, 14, 15 or all of: SFP, EOMES, BRACHYURY, OTX2, ZIC2, ZIC3, ZIC5, DNMT3B, KDR, CDH2, CER1, COL2A1, DAZL, TCF7L1, SOX11, SALL2 or other marker of primed pluripotent state as described herein, including in Table 5.

In any aspect of the present invention, the cell that is undergoing reprogramming is cultured in the second culture conditions or second culture medium for a period of 0.5 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days. Cells are then transferred to the third medium, third culture condition or primed culture medium.

In any aspect of the present invention, the cell that is undergoing reprogramming is cultured in a second culture medium or naïve culture medium for a period of at least about 0.5 days, about 1 day, about 2 days, about 3 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days or about 21 days. Cells are then transferred to the third medium, third culture condition or primed culture medium.

In preferred embodiments of any aspect of the invention, the culturing of the cell in the second culture conditions or second culture medium is for at least 5 days. In a particularly preferred embodiment, the culturing of the cell in the naïve medium (second culture medium or second culture conditions) is for no more than about 21 days.

In any aspect of the present invention, the cell that has been contacted with the second culture medium, or naïve medium, is cultured in a third culture medium or primed culture medium for a period of 0.5 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days or more.

In any aspect of the present invention, the cell that has been contacted with the second culture medium, or naïve medium, is cultured in a third culture medium or primed culture medium for at least about 0.5 day, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days or about 21 days.

It will be understood that any method for reprogramming a somatic cell towards a pluripotent state (i.e., methods for conducting step a) of the first aspect of the invention or steps a) and b) of the second and third aspects of the invention) can be used in accordance with the methods of the present invention. As such, the present invention is not limited by the particular culture conditions for promoting reprogramming towards pluripotency or by the particular method for increasing the protein expression or amount of relevant factors, or culturing conditions to allow the somatic cell to commence reprogramming towards pluripotency. Such methods are known in the art and are further described herein.

In certain embodiments, reprogramming a somatic cell towards a pluripotent state comprises contact the cell with small molecule combinations which induce pluripotency.

In preferred embodiments, the factors for reprogramming the somatic cell towards a pluripotent state are transcription factors.

Any transcription factors associated with pluripotency may be used in accordance with the methods of the present invention. In certain examples, the transcription factors may comprise one or more of, or consist, or consist essentially of the factors: OCT4, SOX2, KLF4 and MYC. In particularly preferred embodiments, the transcription factors comprise all four of the factors OCT4, SOX2, KLF4 and MYC (OSKM), or variants thereof. In further embodiments, the transcription factors for reprogramming the somatic cell, e.g., fibroblast, towards a pluripotent state also comprise the factors LIN28 and/or NANOG. In certain embodiments, the protein expression of each of OCT4, SOX2, KLF4, MYC, LIN28 and NANOG is increased in the somatic cell.

Typically, the protein expression, or amount, of a factor as described herein is increased by contacting the cell with an agent which increases the expression of the factor. Preferably, the agent is selected from the group consisting of: a nucleotide sequence, a protein, an aptamer and small molecule, ribosome, RNAi agent and peptide-nucleic acid (PNA) and analogues or variants thereof. In some embodiments, the agent is exogenous. The present invention also contemplates the use of a transcriptional activation system (e.g., a gRNA for use in a gene regulation system such as CRISPR/Cas9 or TALEN) for increasing the expression of the one or more transcription factors.

Typically, the protein expression, or amount, of a transcription factor as described herein is increased by introducing at least one nucleic acid comprising a nucleotide sequence encoding a transcription factor, or encoding a functional fragment thereof, in the cell.

In a preferred embodiment of the invention, the nucleic acid sequence encoding a transcription factor protein is introduced into a cell by a plasmid. One or more nucleic acids encoding one or more transcription factors may be used. Therefore, it is apparent that one or more plasmids may be used for the purpose of increasing the expression or amount of the required one or more transcription factors. In other words, the nucleic acid sequences may be in or on a single plasmid, or provided to the somatic cell in two or more plasmids.

In any embodiment of the present invention, the plasmid containing the nucleic acid encoding the one or more transcription factors for use according to the invention may be an episomal plasmid.

Preferably, the nucleic acid further includes a heterologous promoter. Preferably, the nucleic acid is in a vector, such as a viral vector or a non-viral vector. Preferably, the vector is a viral vector comprising a genome that does not integrate into the host cell genome. The viral vector may be a retroviral vector, a lentiviral vector, an adenovirus or Sendai virus.

In any embodiment of the invention, the protein expression or amount of the factors is increased in the somatic cell by contacting the somatic cell with one or more agents for increasing the expression of said factors in the cell. In certain embodiments, the protein expression or amount of the factors is increased in the somatic cell by transduction or transfection of the somatic cell with one or more vectors encoding said transcription factors. The vector may be a viral vector, including an integrating or non-integrating viral vector. In further embodiments, the vector may be an episomal vector.

In another aspect, the present invention provides an induced pluripotent stem cell (iPSC), obtained or obtainable by any method of the invention described herein.

In further embodiments, the invention provides an isolated induced pluripotent stem cell (iPSC), obtained or obtainable by any method of the invention described herein.

Further still, the invention provides a population of cells comprising an induced pluripotent stem cell (iPSC), obtained or obtainable by any method of the invention described herein. Preferably, the present invention provides a population of cells, wherein at least 5% of cells are iPSCs and those iPSCs are obtained or obtainable by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population are iPSCs and those iPSCs are obtained or obtainable by a method as described herein.

In any method of the invention, the method further comprises a step of differentiating the iPSC obtained or obtainable by any method of the invention described herein. The step of differentiating the cells may include culturing the iPSCs for a sufficient time and under conditions for generating a cell having at least one characteristic of a differentiated cell or a cell that is not in a pluripotent state.

Accordingly, the invention provides a differentiated cell produced from an induced pluripotent stem cell (iPSC), obtained or obtainable by any method of the invention described herein. The invention also provides an isolated differentiated cell produced from an induced pluripotent stem cell (iPSC), obtained or obtainable by any method of the invention described herein. Further still, the invention provides a population of cells comprising a differentiated cell produced from an induced pluripotent stem cell (iPSC), obtained or obtainable by any method of the invention described herein. Preferably, the present invention provides a population of cells, wherein at least 5% of cells are differentiated cells and those differentiated cells are produced from iPSCs obtained or obtainable by a method as described herein. Preferably, at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population are differentiated cells and those differentiated cells are produced from iPSCs obtained or obtainable by a method as described herein.

The present invention also provides an organoid or other organised collection of cells derived from a population of iPSCs/differentiated cells generated from iPSCs, wherein the iPSCs are obtained or obtainable according to a method of the invention.

The present invention also provides a pharmaceutical composition comprising:
an isolated iPSC or population of iPSCs obtained or obtainable according to a method of the present invention;
an isolated differentiated cell, or population of differentiated cells, derived from one or more iPSCs obtained or obtainable according to a method of the invention; or
an organoid derived from a population of iPSCs/differentiated cells generated from iPSCs, wherein the iPSCs are obtained or obtainable according to a method of the invention;

and a pharmaceutically acceptable excipient.

Further, the present invention provides a method of treating a disease or condition requiring administration of an iPSC or population of cells, comprising administering to a subject in need thereof:
an isolated iPSC or population of iPSCs obtained or obtainable according to a method of the present invention;
an isolated differentiated cell, or population of differentiated cells, derived from one or more iPSCs obtained or obtainable according to a method of the invention; or
an organoid derived from a population of iPSCs/differentiated cells generated from iPSCs, wherein the iPSCs are obtained or obtainable according to a method of the invention;

The present invention also provides a use of an iPSC obtained or obtainable according to a method of the present invention, in the manufacture of a medicament for treating a disease or condition requiring administration of a pluripotent stem cell, or differentiated cell derived therefrom.

The present invention also provides a kit for producing an iPSC according to the methods described herein. Preferably the kit also comprises written instructions for performing a method of the invention. The kit may also comprise one or more of the reagents for performing the methods of the invention, including a first, second and/or third culture medium as defined herein. Further still, the kit may comprise written instructions and/or reagents for differentiating an iPSC produced according to the methods of the invention.

Any method as described herein may have one or more, or all, steps performed in vitro, ex vivo or in vivo.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Acquisition of aberrant DNA methylation occurs after day 13 of primed reprogramming and is absent in naïve iPSCs. a) Table showing the cell lines used to test for differentially methylated regions (DMRs) between iPSCs and ESCs. Columns show culture media used for each cell line used in DMR testing b) Barplots show the number of DMRs detected between iPSCs and ESCs. Memory DMRs are those where no DMR was detected between fibroblasts and iPSCs. iPSC DMRs are those where the methylation level is significantly different between ESCs and fibroblasts. Hyper and hypo-methylated DMRs are those where levels are higher and lower iPSCs compared to ESCs, respectively. c) Plots show mean DNA methylation change across all DMRs compared to the progenitor fibroblast state. Each point represents individual samples. d) Heatmap showing DNA methylation levels in DMRs across all samples for memory and iPSC DMRs. Rows are ordered by correlation clustering. e) Genome browser plot showing DNA methylation levels flanking the transcription start site for TCERG1L and iPSC DMRs. Plots indicate that for this iPSC DMR that aberrant DNA methylation accumulates after day 13 of primed reprogramming and does not appear in naïve reprogramming.

FIG. 2. Naïve-to-primed reprogramming erases somatic cell memory and produces cells that more closely resemble ESCs. a) Schematic representation of naïve-to-primed reprogramming, depicting two distinct strategies that produce transient-naïve-treatment (TNT) primed iPSCs or naïve-to-primed iPSCs. b) Number of CG-DMRs that are corrected by naïve-to-primed and TNT primed reprogramming separated by DMR class. c) Histograms showing the difference in DNA methylation level at DMRs between iPSCs and ESCs, indicating that naïve-to-primed and TNT-primed iPSC reprogramming reduces the DNA methylation difference between iPSCs and ESCs. Vertical dashed lines indicate 20% delta in mCG level, which is the magnitude threshold of DMR significance. d) Barplots show enrichment z-score determined from permutation testing of enrichment of CG-DMRs in repressive chromatin domains. e) Upset plot shows a high degree of CG-DMR correction for DMRs that intersect fibroblast-specific repressive chromatin. f) Upper panel is an aggregate profile plot of CA methylation levels in non-CG DMRs, showing that TNT-primed and naïve-to-primed iPSCs show a non-CG methylation profile highly similar to ESCs. Lower panel shows less H3K9me3 ChIP-seq enrichment in the non-CG DMRs in both TNT-primed iPSCs and naïve-to-primed iPSCs compared to primed iPSCs.

FIG. 3. Naïve-to-primed reprogramming leads to lower transposable element (TE) expression. A) Histograms of DNA methylation levels at TEs indicate DNA methylation is higher in naïve-to-primed iPSCs when compared to primed iPSCs, and more similar to ESCs. Coverage-weighted DNA methylation levels (mCG/CG) were calculated for all copies of each TE. iPSC data shown for cells derived from donor 32F. B) Dendrogram of TE expression indicates naïve-to-primed iPSCs more closely resemble ESCs than primed iPSCs. C) Testing of TE differential expression between primed and naïve-to-primed iPSCs shows TE expression is down-regulated in naïve-to-primed iPSCs. Barplot shows the number of differentially expressed features for genes and TEs. Volcano plot shows the magnitude and significance of expression difference between groups. Dashed vertical lines indicate log-fold change >1 (i.e. absolute fold-change >2). D) The majority of differentially expressed TEs are LINE and LTR elements and are repressed in naïve-to-primed iPSCs. Points indicate the magnitude of change on the y-axis for TEs in each family in the LINE, LTR and SINE TE classes. E) Barplots show normalised TE expression (counts per million) for TEs labelled in (D). In all cases, naïve-to-primed iPSC TE expression is closer to that of H9 ESCs than primed iPSCs. F) Distribution of DNA methylation in transposable element families in DMRs. Methylation is calculated as mCG/CG for each TE for all samples and then grouped by TE family. Shown are corrected DMRs at TEs.

FIG. 4. Differentiation scorecard assessment of embryoid bodies derived from primed and naïve-to-primed iPSCs shows that naïve-to-primed iPSCs exhibit less differentiation bias towards the mesoderm lineage compared to primed iPSCs (fibroblasts are of mesoderm origin and were the donor somatic cell type).

FIG. 5. A) Images show the generation of neural stem cells (NSCs) from primed iPSCs (left) and naïve-to-primed iPSCs (right). This shows that differentiation of primed iPSCs into NSCs produces fibroblast-like cells in the spaces between NSC colonies, which are significantly reduced when differentiating NSCs from naïve-to-primed iPSCs. B) The proportion of cell types detected from single cell RNA-seq experiments for NSC cultures derived from Primed iPSCs, TNT-Primed iPSCs, and Naïve-to-primed iPSCs. Data demonstrate that both Naïve-to-primed and TNT-Primed cells show substantially fewer fibroblast-like cells as characterized by marker gene expression.

FIG. 6. Flow cytometry analyses show the percentage of NCAM positive population on day 8 (D8) and day 16 (D16) of cortical neuron differentiation of MEL1 hESCs, MEL1-derived Primed iPSCs, MEL1-derived TNT-Primed iPSCs, MEL1-derived Naive-to-primed iPSCs (p4) and MEL1-derived Naive-to-primed iPSCs (p10).

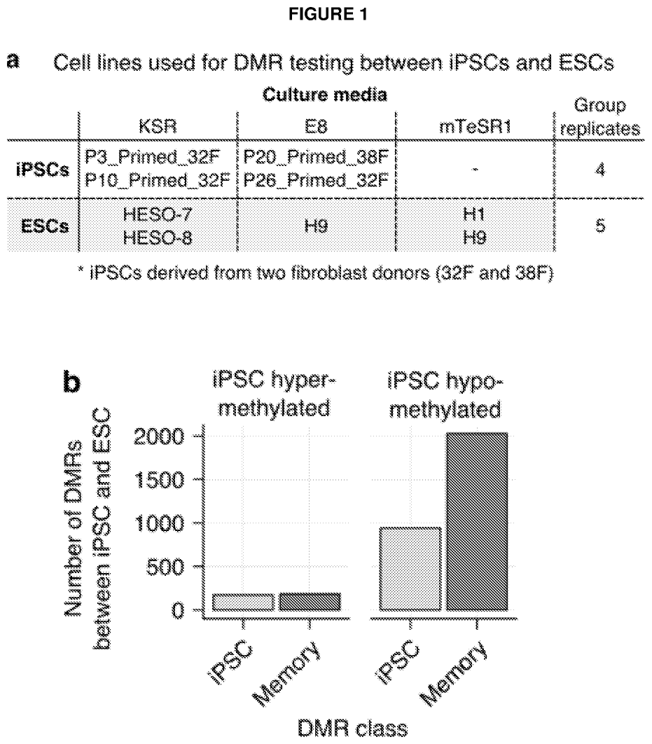

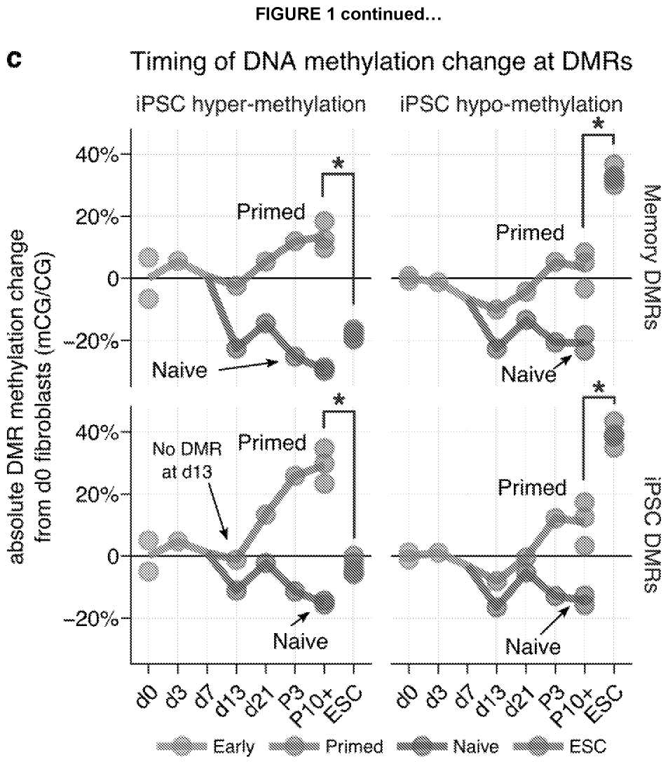

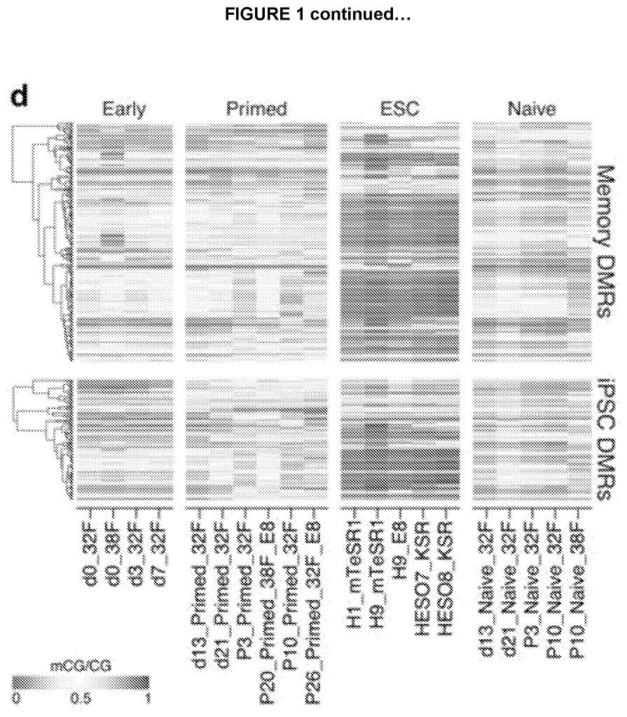

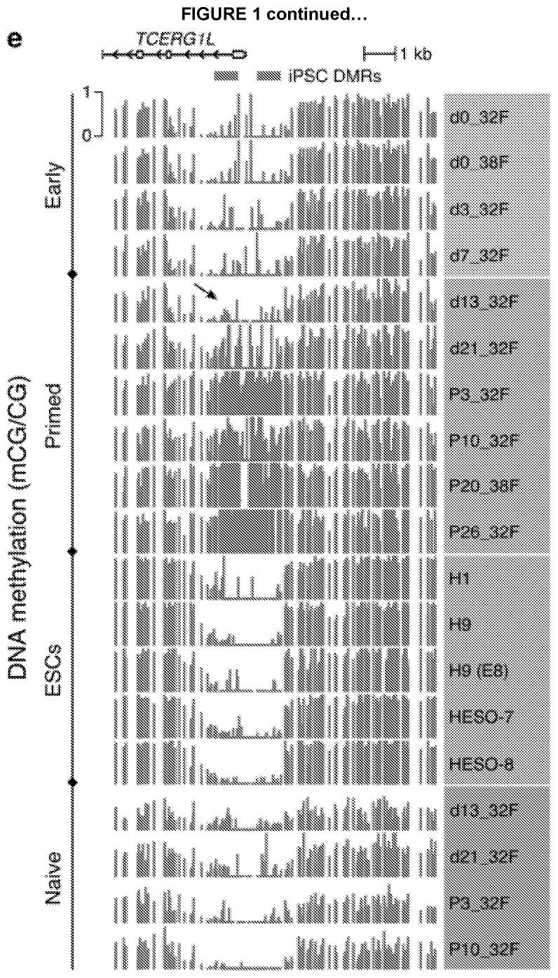

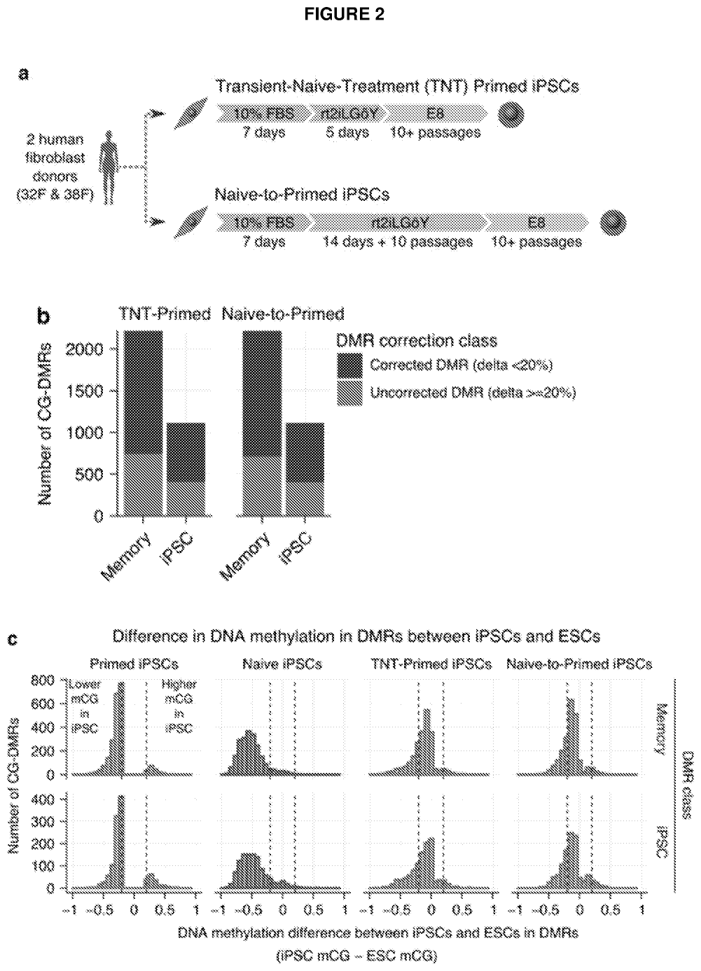

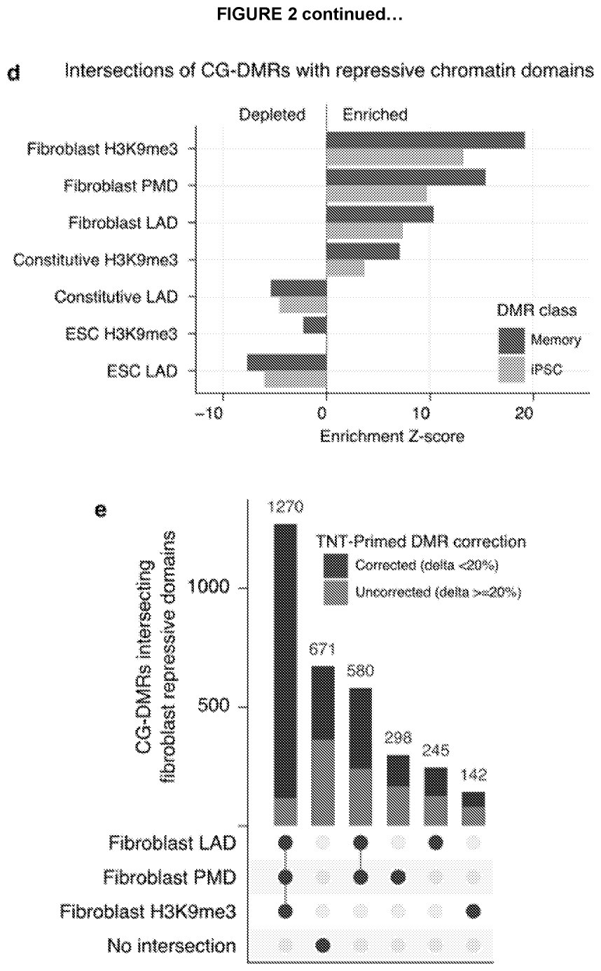

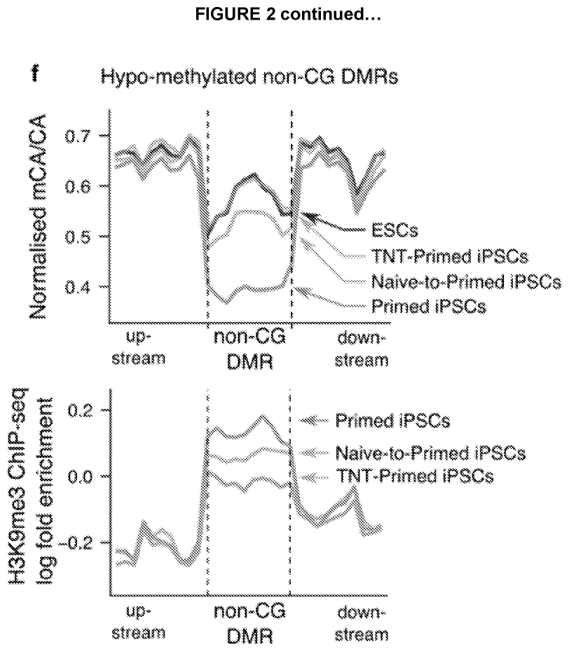

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

For purposes of interpreting this specification, terms used in the singular will also include the plural and vice versa.

According to conventional methods, human induced pluripotent stem cells (iPSCs) and embryonic stem cells (ESCs) are usually maintained in a “primed” state of pluripotency, which resembles post-implantation ESCs. However, these “primed” iPSCs are typically characterised by an epigenomic memory that is distinct from that of ESCs. Further, the primed iPSCs are characterised by the presence of iPSC-specific aberrant DNA methylation states comprising differentially methylated regions (DMRs).

The inventors have comprehensively mapped the pathway of DNA methylation dynamics and patterns during the reprogramming of somatic cells into either primed or naïve iPSCs. Based on this new knowledge, the inventors have developed a novel process which substantially corrects the epigenetic memory and aberrations in the methylation profile of iPSCs. Thus the inventors have developed a new method for generating iPSCs that more closely resemble ESCs in epigenetic and transcriptional characteristics and differentiation potential compared with conventional iPSCs. The iPSCs generated with this new method or process may be referred to herein as “naïve-to-primed iPSCs” (NtoP iPSCs) or “transient-naïve-treatment-primed iPSCs” (TNT-primed iPSCs).

The standard approach for generating human iPSCs, regardless of the method of reprogramming the source (somatic) cell, is by generating iPSCs in primed or naïve media. However, the inventors have developed a process whereby during the reprogramming process, cells are exposed to naïve media, preferably for a short period of time, after which the media is exchanged, and cells placed in a primed media. Thus, the method of the present invention gives rise to iPSCs that are in a primed state (the most commonly used pluripotent state in human iPSCs), but which are more similar to bona fide ESCs compared to iPSCs generated using conventional methods. In particular, they may have one or more, or all, of the following advantages:
significant reduction in the presence of iPSC-specific DMRs (iDMRs);
significant reduction in the presence of memory DMRs;
correction of non-CG methylation in non-CG DMRs to levels similar to ESCs;
significant reduction in the presence of aberrant methylation at transposable elements;
reduction of H3K9me3 ChIP-seq signal in non-CG DMRs, which are associated with fibroblast lamina associated domains (where the original somatic cell type is a fibroblast);
reduced expression of transposable elements compared to conventional primed iPSCs;
reduced differentiation bias into the lineage of the original somatic cell type;
fewer fibroblast-like cells appearing during the differentiation of naïve-to-primed and TNT-primed iPSCs into neuronal stem cells (NSCs) when compared to primed cells (where the original somatic cell type is a fibroblast);
similar differentiation efficiencies into different cell types as genetically matched ESCs.

Cells

As used herein, the term “pluripotent” or “pluripotency” refers to cells with the ability to give rise to progeny that can undergo differentiation, under the appropriate conditions, into cell types that collectively demonstrate characteristics associated with cell lineages from all of the three germinal layers (endoderm, mesoderm, and ectoderm). Pluripotent stem cells can contribute to many or all tissues of a prenatal, postnatal or adult animal. A standard test that is accepted in the art, such as the ability to form a teratoma in 8-12 week old SCID mice, can be used to establish the pluripotency of a cell population, however identification of various pluripotent stem cell characteristics can also be used to detect pluripotent cells.

As used herein, reference to “pluripotent state”, “pluripotent stem cell characteristics” or “one or more characteristics of a pluripotent cell”, refers to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least one, two, or three, and optionally all, of the markers from the following non-limiting list: SSEA-3, SSEA-4, TRA-1-60, TRA-1-81, TRA-2-49/6E, ALP, SO2, E-cadherin, UTF-1, OCT4 (POU5F1), REX1, and NANOG.

Cell morphologies associated with pluripotent stem cells are also pluripotent stem cell characteristics. Pluripotent cells are typically characterised by the capacity for self-renewal, the ability to give rise to cell type of the three germ layers and the expression of pluripotent markers such as OCT4 (POU5F1), NANOG and SOX2. Pluripotent cells typically grow in colonies which are flat with clear borders (when in the primed state) or dome-shaped (when in the naïve state). This can be contrasted with the morphology of somatic cells, for example, fibroblasts which are large and elongated. Markers expressed by both naïve and primed pluripotent cells include: OCT4 (POU5F1), SOX2, NANOG, KLF4, EPCAM, and PRDM14.

Markers which are expressed only by naïve pluripotent cells or only expressed by primed pluripotent cells are listed in Table 5 herein.

As used herein, the term “stem cell” refers to a cell which is not terminally differentiated, i.e., it is capable of differentiating into other cell types having a more particular, specialised function. The term encompasses embryonic stem cells, fetal stem cells, adult stem cells or committed/progenitor cells.

As used herein, an organoid is a collection of organ-specific cell types that develops from stem cells or organ progenitors, self-organizes through cell sorting and spatially restricted lineage commitment in a manner similar to in vivo, and exhibits properties including: multiple organ-specific cell types; is capable of recapitulating some specific function of the organ (e.g. contraction, neural activity, endocrine secretion, filtration, excretion); its cells are grouped together and spatially organized, similar to an organ. Organoids may be used as a tool to study basic biological processes including various disease processes, drug screening, and response to different environmental stimuli.

The terms “CG” or “CpG” can be used interchangeably and refer to regions of a DNA molecule where a cytosine nucleotide occurs upstream of a guanine nucleotide in the linear sequence of bases (linear strand) within the DNA molecule. Nucleotides forming a linear strand in a DNA molecule are linked through a phosphate. Therefore, a CG site is also referred to as a CpG site. The CpG notation is further used to distinguish the linear sequence of cytosine and guanine from the CG base-pairing of cytosine and guanine, where cytosine and guanine are located on opposite strands of a DNA molecule. Cytosine in CpG dinucleotides can be methylated to form 5-methylcytosine. In mammals, methylating the cytosine within a gene may turn the gene off. Enzymes that add a methyl group to a cytosine within a DNA molecule are referred to as DNA methyltransferases.

A non-CpG site is a linear sequence of a DNA molecule in which a cytosine nucleotide occurs upstream of a nucleotide that is not a guanine. Non-CpG cytosine methylation can occur at cytosine sites in which the nucleotide does not form part of a CG dinucleotide sequence.

DNA methylation is an epigenetic mark associated with gene silencing, which mainly occurs at cytosine nucleotides when found adjacent to guanine (CpG sites). CpG sites show clustering in specific genomic regions. These clusters of high CpG density are known as CpG islands, and are often found in gene promoter regions. Although the exact mechanism is yet to be defined, there is evidence that methylation provides a “memory” mark of gene silencing, whilst blocking of transcription is carried out by associated histone modifications.

CpG sites are also found in noncoding genomic regions of DNA and within gene bodies. Here, methylation plays an important role in DNA stabilization and preventing movement of transposable elements such as LINE1. LINE1 is a retrotransposon found in abundance in the human genome, with approximately 500,000 copies, corresponding to 17.88% of the genome. Owing to its abundance in the genome, LINE1 methylation has been validated as a surrogate measure for global DNA methylation.

Prior art methods for generating iPSCs may result in CpG sites that comprise hyper- or hypo-methylated DMRs as compared to the genome of an ESC. A non-CpG hypermethylated DMR refers to a differentially methylated region (DMR) of a primed iPSC genome (wherein the iPSC is obtained by methods of the prior art) having a greater number of methylated non-CpG sites relative to the corresponding region of a human ESC. A non-CpG hypomethylated DMR, refers to a differentially methylated region (DMR) of a primed iPSC genome (wherein the iPSC is obtained by methods of the prior art) having fewer methylated non-CpG sites relative to the corresponding region of a human ESC. The non-CpG hypomethylated or hypermethylated DMRs are typically about 100 bp to 4000 kb in length.

Similarly, prior art methods for generating iPSCs may result in hypermethylated CG-DMRs and hypomethylated CG-DMRs. The CpG hypermethylated or hypomethylated DMR is typically about 100 bp to 4000 kb in length.

It will be understood that the methods of the present invention enable the correction of at least one or more of the aberrant DMRs observed in iPSCs obtained using conventional methods.

In any aspect of the invention, the iPSCs obtained or obtainable by the methods of the invention have a level of CpG DNA methylation, level of non-CpG methylation and/or level of CpG DNA methylation for a transposable element that more closely resembles the DNA methylation profile at the same genetic locus for an ESC. In some embodiments, the genetic locus is one or more of the regions defined in any of Tables 3 or 4, herein.

In any embodiment, the methods of the present invention provide for iPSCs that have a level of methylation at a LINE1 retrotransposon that is the same, or substantially the same as in an ESC.

Non-limiting examples of CpG hyper- or hypomethylated DMRs associated with incomplete iPSC reprogramming are described in U.S. Pat. No. 9,428,811, incorporated herein by reference. It will be appreciated that one or more of the DMRs described in U.S. Pat. No. 9,428,811 are corrected by reprogramming somatic cells in accordance with the present invention.

Methods for identifying epigenomic signatures of a cell relative to the equivalent signature of an ESC are also described in U.S. Pat. No. 9,428,811 and can be used in accordance with the methods of the present invention to confirm that the iPSCs generated (or intermediates obtained during the process of the methods of the invention), have similar epigenomic profiles to ESCs. More specifically, the skilled person can determine the epigenomic profile of a cell that has been contacted with the second culture medium, as described herein, to confirm that the cell has been hypomethylated, or transitioned to an epigenomic state that resembles that of an ESC, and can be transitioned into the third culture medium.

In preferred embodiments of the present invention, a somatic cell is reprogrammed towards a pluripotent state, including by culturing the cell in a second culture condition or culture medium adapted to promote hypomethylation. The conditions may include use of media which promote a naïve pluripotent state. However, it will be understood that prior to the cells reaching a naïve pluripotent state, the cells are preferably transitioned into a third culture condition/medium to promote a primed pluripotent state.

Thus, in preferred embodiments, the cells that have been contacted with the second culture medium, or exposed to the second culture condition, as herein described, have a genome-wide hypomethylation prior to being contacted with the third culture medium. Preferably the genome-wide hypomethylation is 70%, 60%, 50%, 40%, 30% or less than the methylation state of a primed pluripotent stem cell, preferably 50% or less.

Hypomethylation is a distinctive property of primitive cells in human embryos that is lost in conventional (primed) PSCs. Cells may show global demethylation in response to being transiently exposed to naïve media, in accordance with the method of the invention. Alternatively, the cells may show a reduction of at least 10-90%, preferably about at least 50% in methylation of CpG genome-wide. Methylation levels may be measured in comparison to methylation of primed iPSCs that have not been contacted with naïve media. Methylation levels may be quantified by nucleoside mass spectrometry or by bisulfite conversion coupled to deep sequencing.

Cells that are contacted with the second culture medium, in accordance with the present invention, may also have lower levels of histone modifications associated with gene silencing, such as H3K9me3, compared with primed iPSCs that have not been contacted with the second culture medium. Levels of histone modifications may be measured by quantitative immunostaining or chromatin immunoprecipitation coupled to deep sequencing (ChIP-seq).

Female cells in the naïve state may show reversible epigenetic erasure of the X chromosome, more specifically demethylation of the X chromosome and absence of H3K27me3 foci in XX naïve cells, preferably in 80% or more the XX cells. In some embodiments, conversion of cells from naïve to primed state restores H3K27me3 foci in the majority of cells, preferably by at least 50%, 60%, 70% or more.

The skilled person will be familiar with the terms “naïve” and “primed” in respect of stem cells. These terms were identified more than a decade ago in order to describe early and late phases of epiblast ontogeny and to describe ESC and EpiSC derivatives. As used herein, a naïve pluripotent state refers to a pluripotent state that more closely resembles the pre-implantation embryo. In some circumstances, the term “naïve state” is used interchangeably with the term “ground state”. Naïve state cells are a stable self-renewing culture of homogeneous pluripotent stem cells that are substantially epigenetically reset compared to somatic cells and have the developmental identity and functional capacity of pre-implantation epiblast.

In certain embodiments, a naïve pluripotent cell may express mRNA and protein of pre-implantation epiblast specific transcription factors. For example, naïve cells may express 1, 2, 3, 4, 5, 6, 7, 8, 9, 10, 11, 12, 13, 14, 15, 16, 17, 18, 19, 20, 21, 22, 23, 24, 25 or all of: KLF2, KLF4, TFCP2L1, TBX3, REX1, GBX2, STELLA (DPPA3), KLF17, DPPA3, DPPA5, TFCP2L1, MAEL, UTF1, ZFP57, DNMT3L, FGF4, FOXR1, ARGFX, TRIM60, DDX43, BRDT, ALPPL2, KHDC3L, KHDC1L and PRAP1. Other markers indicative of a naïve pluripotent state are shown in Table 5.

In certain embodiments, a primed pluripotent cell may express mRNA and protein of post-implantation epiblast specific transcription factors. For example, primed cells may express 1, 2, 3, 4, 5, 6, 7, 8, 9, 10, 11, 12, 13, 14, 15 or all of: SFP, EOMES, BRACHYURY, OTX2, ZIC2, ZIC3, ZIC5, DNMT3B, KDR, CDH2, CER1, COL2A1, DAZL, TCF7L1, SOX11, SALL2. Other markers indicative of a primed pluripotent state are shown in Table 5.

Since identifying naïve and primed pluripotent states, a number of different culture media have been developed which are suitable for sustaining pluripotent cells in either state. The skilled person will be familiar with such media, examples of which are provided further herein.

Still further, naïve cells may be characterised by their morphology. A naïve phenotype is typically characterised by tightly-packed, round, domed appearance. The cells may also be described as forming compact refractile colonies. In accordance with a preferred embodiment of the present invention, cells that are cultured in the second culture media have not developed a morphology that is typical of naïve cells when they are transitioned into the third culture medium.

As used herein, “naïve pluripotent state” or “naïve pluripotent phenotype” will be understood to refer to a cell phenotype that includes cells that are round, dome-shaped. As used herein, a naïve pluripotent state refers to refers to a pluripotent states that more closely resembles the pre-implantation embryo.

As used herein, a “primed pluripotent state” or “primed pluripotent phenotype” typically refers to a cell phenotype characterised by the presence of flat cell colonies with clear borders. As used herein, a primed pluripotent state refers to a pluripotent states that more closely resembles the epiblast of the post-implantation embryo.

An “induced pluripotent stem cell” refers to a pluripotent stem cell artificially derived from a non-pluripotent cell.

As used herein, “non-pluripotent cells” refer to mammalian cells that are not pluripotent cells. The terms “non-pluripotent cell”, “somatic cell” and “differentiated cell” may be used interchangeably. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells.

It will be understood that in accordance with the methods of the invention, any non-pluripotent cell (i.e., any differentiated or somatic cell) can be used as a starting cell from which to generate an iPSC. Further, the iPSC obtainable or obtained according to the present invention can be differentiated into a non-pluripotent state.

The somatic cell may be an adult cell or a cell derived from an adult which displays one or more detectable characteristics of an adult or non-embryonic cell. The diseased cell may be a cell displaying one or more detectable characteristics of a disease or condition.

As used herein, a “somatic cell” refers to a terminally differentiated cell. As used herein, the term “somatic cell” refers to any cell forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. The somatic cells may be immortalized to provide an unlimited supply of cells, for example, by increasing the level of telomerase reverse transcriptase (TERT). For example, the level of TERT can be increased by increasing the transcription of TERT from the endogenous gene, or by introducing a transgene through any gene delivery method or system.

Differentiated somatic cells, including cells from a fetal, newborn, juvenile or adult primate, including human, individual, are suitable somatic cells in the methods of the invention. Suitable somatic cells include, but are not limited to, bone marrow cells, epithelial cells, endothelial cells, fibroblast cells, hematopoietic cells, keratinocytes, hepatic cells, intestinal cells, mesenchymal cells, myeloid precursor cells and spleen cells. Alternatively, the somatic cells can be cells that can themselves proliferate and differentiate into other types of cells, including blood stem cells, muscle/bone stem cells, brain stem cells and liver stem cells. Suitable somatic cells are receptive, or can be made receptive using methods generally known in the scientific literature, to uptake of transcription factors including genetic material encoding the transcription factors. Uptake-enhancing methods can vary depending on the cell type and expression system. Exemplary conditions used to prepare receptive somatic cells having suitable transduction efficiency are well-known by those of ordinary skill in the art. The starting somatic cells can have a doubling time of about twenty-four hours.

In preferred embodiments, the somatic cell is a fibroblast (preferably a dermal fibroblast or a cardiac), keratinocytes (preferably epidermal keratinocytes), a monocyte or an endothelial cell.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein, refers to a population of cells that has been removed and separated from a mixed or heterogeneous population of cells. In some embodiments, an isolated population is a substantially pure population of cells as compared to the heterogeneous population from which the cells were isolated or enriched from.

The skilled person will also be able to determine when a somatic cell has commenced reprogramming towards a pluripotent state when the somatic cell loses morphological characteristics that are typical of the somatic cell. Again, the skilled person will be familiar with morphological characteristics for somatic cells, including fibroblasts.

A somatic cell may also be determined to be reprogrammed to a pluripotent state, when it displays at least one characteristic of a pluripotent cell. The one or more characteristics of a pluripotent cell (e.g., an iPSC or ESC) include up-regulation of any one or more ESC markers and/or changes in cell morphology. Typically, a cell that is converted to an iPSC/ESC-like cell will display 1, 2, 3, 4, 5, 6, 7, 8 or more characteristics of the iPSC/ESC.

In some embodiments where an individual is to be treated with the resulting pluripotent cells, or differentiated cells derived therefrom, the individual's own non-pluripotent cells are used to generate pluripotent cells according to the methods of the invention.

Cells can be from, e.g., humans or non-human mammals. Exemplary non-human mammals include, but are not limited to, mice, rats, cats, dogs, rabbits, guinea pigs, hamsters, sheep, pigs, horses, and bovines.

### Reprogramming

The skilled person will be familiar with standard techniques for promoting reprogramming of a somatic cell towards a pluripotent state.

Various methods for reprogramming a somatic cell towards a pluripotent state are known in the art. Reprogramming of somatic cells typically involves the expression of reprogramming factors (including transcription factors), followed by culture in particular conditions for promoting the loss of markers of differentiation, and the gain of pluripotency markers.

Examples of suitable methods for reprogramming somatic cells are replete in the art, and are exemplified in WO 2009/101407, WO 2014/200030, WO 2015/056804, WO 2014/200114, WO 2014/065435, WO 2013/176233, WO 2012/060473, WO 2012/036299, WO 2011/158967, WO 2011/055851, WO 2011/037270, WO 2011/090221, the contents of which are hereby incorporated by reference.

Particularly preferred transcription factors, and nucleic acid sequences thereof, that may be used to reprogram a somatic cell (e.g., a fibroblast) in accordance with the methods of the invention are shown below in Table 1. It will be understood however that the present invention is not limited to the use of the transcription factors recited in Table 1 in order to reprogram a somatic cell.

The transcription factors and other protein factors referred to herein are referred to by the HUGO Gene Nomenclature Committee (HGNC) Symbol. Table 1 provides exemplary Ensemble Gene ID and Uniprot IDs for the transcription factors recited herein. The nucleotide sequences are derived from the Ensembl database (Flicek et al. (2014). Nucleic Acids Research Volume 42, Issue D1. Pp. D749-D755) version 83. Also contemplated for use in the invention is any variant, homolog, ortholog or paralog of a transcription factor referred to herein.

The skilled person will also be familiar with the ability to reprogram somatic cells towards a naïve pluripotent state (as compared to a primed pluripotent state). It will be understood that the methods of the present invention apply to cells that have been treated so as to promote reprogramming towards a naïve or primed pluripotent state. In preferred embodiments of the invention, the method comprises increasing the protein expression of one or more factors in the somatic cell for reprogramming the somatic cell towards a naïve pluripotent state.

The skilled person will appreciate that this information may be used in performing the methods of the present invention, for example, for the purposes of providing increased amounts of transcription factors in somatic cells, or providing nucleic acids (including recombinant polynucleotides) or the like for recombinantly expressing a transcription factor in a somatic cell.

TABLE 1
Accession numbers identifying exemplary nucleotide sequences and
amino acid sequences of transcription factors referred to herein.

| Transcription factor Associated Gene | | |
|---|---|---|
| Name | Ensembl Gene ID | Uniprot ID |
| OCT4 (also called POU5F1) | ENSG00000204531 | Q01860 |
| SOX2 | ENSG00000181449 | P48431 |
| cMYC | ENSG00000136997 | P01106 |
| KLF4 | ENSG00000136826 | O43474 |
| LIN28 | ENSG00000131914 | Q9H9Z2 |
| NANOG | ENSG00000111704 | Q9H9S0 |

The present invention contemplates the use of variants of the transcription factors described herein. The variant could be a fragment of full length polypeptide or a naturally occurring splice variant. The variant could be a polypeptide at least 70%, 80%, 85%, 90%, 95%, 98%, or 99% identical to a fragment of the polypeptide, wherein the fragment is at least 50%, 60%, 70%, 80%, 85%, 90%, 95%, 98%, or 99% as long as the full length wild type polypeptide or a domain thereof has a functional activity of interest such as the ability to promote conversion of a somatic cell type to a target cell type. In some embodiments the domain is at least 100, 200, 300, or 400 amino acids in length, beginning at any amino acid position in the sequence and extending toward the C-terminus. Variations known in the art to eliminate or substantially reduce the activity of the protein are preferably avoided. In some embodiments, the variant lacks an N- and/or C-terminal portion of the full length polypeptide, e.g., up to 10, 20, or 50 amino acids from either terminus is lacking. In some embodiments the polypeptide has the sequence of a mature (full length) polypeptide, by which is meant a polypeptide that has had one or more portions such as a signal peptide removed during normal intracellular proteolytic processing (e.g., during co-translational or post-translational processing). In some embodiments wherein the protein is produced other than by purifying it from cells that naturally express it, the protein is a chimeric polypeptide, by which is meant that it contains portions from two or more different species. In some embodiments wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. For example, the ability of a variant of a transcription factor to convert a somatic cell to a target cell type can be assessed using the assays as disclosed herein in the Examples. Other convenient assays include measuring the ability to activate transcription of a reporter construct containing a transcription factor binding site operably linked to a nucleic acid sequence encoding a detectable marker such as luciferase. In certain embodiments of the invention a functional variant or fragment has at least 50%, 60%, 70%, 80%, 90%, 95% or more of the activity of the full length wild type polypeptide.

The term “increasing the amount of” with respect to increasing an amount of a transcription factor, refers to increasing the quantity of the transcription factor in a cell of interest (e.g., a somatic cell such as a fibroblast). In some embodiments, the amount of transcription factor is “increased” in a cell of interest (e.g., a cell into which an expression cassette directing expression of a polynucleotide encoding one or more transcription factors has been introduced) when the quantity of transcription factor is at least 10%, 20%, 30%, 40%, 50%, 60%, 70%, 80%, 90%, or more relative to a control (e.g., a fibroblast into which none of said expression cassettes have been introduced). However, any method of increasing an amount of a transcription factor is contemplated including any method that increases the amount, rate or efficiency of transcription, translation, stability or activity of a transcription factor (or the pre-mRNA or mRNA encoding it). In addition, down-regulation or interference of a negative regulator of transcription expression, increasing efficiency of existing translation (e.g. SINEUP) are also considered.

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

It will be appreciated that where two or more proteins are to be expressed in a cell, one or multiple expression cassettes can be used. For example, where one expression cassette is to express multiple polypeptides, a polycistronic expression cassette can be used.

In some embodiments, expression cassettes encoding reprogramming transcription factor proteins can be introduced into cells as part of a vector. Exemplary vectors include, e.g., plasmids, and viral vectors (including but not limited to adenoviral, AAV, or retroviruses such as lentiviruses).

Nucleic Acids and Vectors

A nucleic acid or vector comprising a nucleic acid as described herein may include one or more of the sequences referred to above in Table 1.

The term “expression” refers to the cellular processes involved in producing RNA and proteins and as appropriate, secreting proteins, including where applicable, but not limited to, for example, transcription, translation, folding, modification and processing.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “vector” refers to a carrier DNA molecule into which a DNA sequence can be inserted for introduction into a host or somatic cell. Preferred vectors are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector. Vectors can be viral vectors or non-viral vectors. Should viral vectors be used, it is preferred the viral vectors are replication defective, which can be achieved for example by removing all viral nucleic acids that encode for replication. A replication defective viral vector will still retain its infective properties and enters the cells in a similar manner as a replicating adenoviral vector, however once admitted to the cell a replication defective viral vector does not reproduce or multiply. Vectors also encompass liposomes and nanoparticles and other means to deliver DNA molecule to a cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. The term “operatively linked” includes having an appropriate start signal (e.g. ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

The term “viral vectors” refers to the use of viruses, or virus-associated vectors as carriers of a nucleic acid construct into a cell. Constructs may be integrated and packaged into non-replicating, defective viral genomes like Adenovirus, Adeno-associated virus (AAV), or Herpes simplex virus (HSV) or others, including retroviral and lentiviral vectors, for infection or transduction into cells. The vector may or may not be incorporated into the cell's genome. The constructs may include viral sequences for transfection, if desired. Alternatively, the construct may be incorporated into vectors capable of episomal replication, e.g EPV and EBV vectors.

As used herein, the term “adenovirus” refers to a virus of the family Adenovirida. Adenoviruses are medium-sized (90-100 nm), nonenveloped (naked) icosahedral viruses composed of a nucleocapsid and a double-stranded linear DNA genome.

As used herein, the term “non-integrating viral vector” refers to a viral vector that does not integrate into the host genome; the expression of the gene delivered by the viral vector is temporary. Since there is little to no integration into the host genome, non-integrating viral vectors have the advantage of not producing DNA mutations by inserting at a random point in the genome. For example, a non-integrating viral vector remains extra-chromosomal and does not insert its genes into the host genome, potentially disrupting the expression of endogenous genes. Non-integrating viral vectors can include, but are not limited to, the following: adenovirus, alphavirus, picornavirus, and vaccinia virus. These viral vectors are “non-integrating” viral vectors as the term is used herein, despite the possibility that any of them may, in some rare circumstances, integrate viral nucleic acid into a host cell's genome. What is critical is that the viral vectors used in the methods described herein do not, as a rule or as a primary part of their life cycle under the conditions employed, integrate their nucleic acid into a host cell's genome.

The vectors described herein can be constructed and engineered using methods generally known in the scientific literature to increase their safety for use in therapy, to include selection and enrichment markers, if desired, and to optimize expression of nucleotide sequences contained thereon. The vectors should include structural components that permit the vector to self-replicate in the somatic cell type. For example, the known Epstein Barr oriP/Nuclear Antigen-1 (EBNA-I) combination (see, e.g., Lindner, S. E. and B. Sugden, The plasmid replicon of Epstein-Barr virus: mechanistic insights into efficient, licensed, extrachromosomal replication in human cells, Plasmid 58:1 (2007), incorporated by reference as if set forth herein in its entirety) is sufficient to support vector self-replication and other combinations known to function in mammalian, particularly primate, cells can also be employed. Standard techniques for the construction of expression vectors suitable for use in the present invention are well-known to one of ordinary skill in the art and can be found in publications such as Sambrook J, et al., “Molecular cloning: a laboratory manual,” (3rd ed. Cold Spring harbor Press, Cold Spring Harbor, N.Y. 2001), incorporated herein by reference as if set forth in its entirety.

In the methods of the invention, genetic material encoding the relevant transcription factors required for a conversion is delivered into the somatic cells via one or more reprogramming vectors. Each transcription factor can be introduced into the somatic cells as a polynucleotide transgene that encodes the transcription factor operably linked to a heterologous promoter that can drive expression of the polynucleotide in the somatic cell.

Suitable reprogramming vectors are any described herein, including episomal vectors, such as plasmids, that do not encode all or part of a viral genome sufficient to give rise to an infectious or replication-competent virus, although the vectors can contain structural elements obtained from one or more virus. One or a plurality of reprogramming vectors can be introduced into a single somatic cell. One or more transgenes can be provided on a single reprogramming vector. One strong, constitutive transcriptional promoter can provide transcriptional control for a plurality of transgenes, which can be provided as an expression cassette. Separate expression cassettes on a vector can be under the transcriptional control of separate strong, constitutive promoters, which can be copies of the same promoter or can be distinct promoters. Various heterologous promoters are known in the art and can be used depending on factors such as the desired expression level of the transcription factor. It can be advantageous, as exemplified below, to control transcription of separate expression cassettes using distinct promoters having distinct strengths in the cells. Another consideration in selection of the transcriptional promoters is the rate at which the promoter(s) is silenced. The skilled artisan will appreciate that it can be advantageous to reduce expression of one or more transgenes or transgene expression cassettes after the product of the gene(s) has completed or substantially completed its role in the reprogramming method. Exemplary promoters are the human EF1α elongation factor promoter, CMV cytomegalovirus immediate early promoter and CAG chicken albumin promoter, and corresponding homologous promoters from other species. In human somatic cells, both EF1α and CMV are strong promoters, but the CMV promoter is silenced more efficiently than the EF1α promoter such that expression of transgenes under control of the former is turned off sooner than that of transgenes under control of the latter. The transcription factors can be expressed in the somatic cells in a relative ratio that can be varied to modulate reprogramming efficiency. Preferably, where a plurality of transgenes is encoded on a single transcript, an internal ribosome entry site is provided upstream of transgene(s) distal from the transcriptional promoter. Although the relative ratio of factors can vary depending upon the factors delivered, one of ordinary skill in possession of this disclosure can determine an optimal ratio of factors.

The skilled artisan will appreciate that the advantageous efficiency of introducing all factors via a single vector rather than via a plurality of vectors, but that as total vector size increases, it becomes increasingly difficult to introduce the vector. The skilled artisan will also appreciate that position of a transcription factor on a vector can affect its temporal expression, and the resulting reprogramming efficiency. As such, Applicants employed various combinations of factors on combinations of vectors. Several such combinations are here shown to support reprogramming.

After introduction of the reprogramming vector(s) and while the somatic cells are being reprogrammed, the vectors can persist in target cells while the introduced transgenes are transcribed and translated. Transgene expression can be advantageously downregulated or turned off in cells that have been reprogrammed to a target cell type. The reprogramming vector(s) can remain extra-chromosomal. At extremely low efficiency, the vector(s) can integrate into the cells' genome. The examples that follow are intended to illustrate but in no way limit the present invention.

Suitable methods for nucleic acid delivery for transformation of a cell, a tissue or an organism for use with the current invention are believed to include virtually any method by which a nucleic acid (e.g., DNA) can be introduced into a cell, a tissue or an organism, as described herein or as would be known to one of ordinary skill in the art (e.g., Stadtfeld and Hochedlinger, Nature Methods 6(5):329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van Der Eb, Virology, 52:456-467, 1973; Chen and Okayama, Mol. Cell Biol., 7(8):2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference.

A number of polypeptides capable of mediating introduction of associated molecules into a cell have been described previously and can be adapted to the present invention. See, e.g., Langel (2002) Cell Penetrating Peptides: Processes and Applications, CRC Press, Pharmacology and Toxicology Series. Examples of polypeptide sequences that enhance transport across membranes include, but are not limited to, the Drosophila homeoprotein antennapedia transcription protein (AntHD) (Joliot et al., New Biol. 3: 1121-34, 1991; Joliot et al., Proc. Natl. Acad. Sci. USA, 88: 1864-8, 1991; Le Roux et al., Proc. Natl. Acad. Sci. USA, 90: 9120-4, 1993), the herpes simplex virus structural protein VP22 (Elliott and O'Hare, Cell 88: 223-33, 1997); the HIV-1 transcriptional activator TAT protein (Green and Loewenstein, Cell 55: 1179-1188, 1988; Frankel and Pabo, Cell 55: 1 289-1193, 1988); Kaposi FGF signal sequence (kFGF); protein transduction domain-4 (PTD4); Penetratin, M918, Transportan-10; a nuclear localization sequence, a PEP-I peptide; an amphipathic peptide (e.g., an MPG peptide); delivery enhancing transporters such as described in U.S. Pat. No. 6,730,293 (including but not limited to a peptide sequence comprising at least 5-25 or more contiguous arginines or 5-25 or more arginines in a contiguous set of 30, 40, or 50 amino acids; including but not limited to a peptide having sufficient, e.g., at least 5, guanidino or amidino moieties); and commercially available Penetratin™ 1 peptide, and the Diatos Peptide Vectors (“DPVs”) of the Vectocell® platform available from Daitos S. A. of Paris, France. See also, WO/2005/084158 and WO/2007/123667 and additional transporters described therein. Not only can these proteins pass through the plasma membrane but the attachment of other proteins, such as the transcription factors described herein, is sufficient to stimulate the cellular uptake of these complexes.

Agents

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclic moieties including macrolides, leptomycins and related natural products or analogues thereof. Compounds can be known to have a desired activity and/or property, or can be selected from a library of diverse compounds.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells, or is otherwise flanked by a different nucleic acid sequence than that found in nature. An exogenous nucleic acid may also be extra-chromosomal, such as an episomal vector.

Screening one or more candidate agents for the ability to increase the amount of the one or more transcription factors required for reprogramming of a somatic cell type to a pluripotent state may include the steps of contacting a system that allows the product or expression of a transcription factor with the candidate agent and determining whether the amount of the transcription factor has increased. The system may be in vivo, for example a tissue or cell in an organism, or in vitro, a cell isolated from an organism or an in vitro transcription assay, or ex vivo in a cell or tissue. The amount of transcription factor may be measured directly or indirectly, and either by determining the amount of protein or RNA (e.g. mRNA or pre-mRNA). The candidate agent function to increase the amount of a transcription factor by increasing any step in the transcription of the gene encoding the transcription factor or increase the translation of corresponding mRNA. Alternatively, the candidate agent may decrease the inhibitory activity of a repressor of transcription of the gene encoding the transcription factor or the activity of a molecule that causes the degradation of the mRNA encoding the transcription factor or the protein of the transcription factor itself.

Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Such labelled reagents may be used in a variety of well-known assays, such as radioimmunoassays, enzyme immunoassays, e.g., ELISA, fluorescent immunoassays, and the like. See, for example, U.S. Pat. Nos. 3,766,162; 3,791,932; 3,817,837; and 4,233,402.

The methods of the invention include high-throughput screening applications. For example, a high-throughput screening assay may be used which comprises any of the assays according to the invention wherein aliquots of a system that allows the product or expression of a transcription factor are exposed to a plurality of candidate agents within different wells of a multi-well plate. Further, a high-throughput screening assay according to the disclosure involves aliquots of a system that allows the product or expression of a transcription factor which are exposed to a plurality of candidate agents in a miniaturized assay system of any kind.

The method of the disclosure may be “miniaturized” in an assay system through any acceptable method of miniaturization, including but not limited to multi-well plates, such as 24, 48, 96 or 384-wells per plate, microchips or slides. The assay may be reduced in size to be conducted on a micro-chip support, advantageously involving smaller amounts of reagent and other materials. Any miniaturization of the process which is conducive to high-throughput screening is within the scope of the invention.

In any method of the invention the iPSCs, or differentiated cells derived therefrom, can be transferred into the same mammal from which the somatic cells were obtained. In other words, the somatic cells used in a method of the invention can be an autologous cell, i.e., can be obtained from the same individual in which the iPSCs or differentiated cells derived therefrom are to be administered. Alternatively, the iPSCs cell can be allogenically transferred into another individual. Preferably, the cell is autologous to the subject in a method of treating or preventing a medical condition in the individual.

Culturing of Cells

Cells to be induced to pluripotency can be cultured according to any method known in the art. General guidelines can be found in, e.g., Maherali, et al., Cell Stem Cell 3:595-605 (2008).

In some embodiments, the cells are cultured in contact with feeder cells. Exemplary feeder cells include, but are not limited to fibroblast cells, e.g., mouse embryonic fibroblast (MEF) cells. Methods of culturing cells on feeder cells is known in the art.

In some embodiments, the cells are cultured in the absence of feeder cells. Cells, for example, can be attached directly to a solid culture surface (e.g., a culture plate), e.g., via a molecular tether. The inventors have found that culturing cells induced to pluripotency have a much greater efficiency of induction to pluripotency (i.e., a greater portion of cells achieve pluripotency) when the cells are attached directly to the solid culturing surface compared the efficiency of otherwise identically-treated cells that are cultured on feeder cells. Exemplary molecular tethers include, but are not limited to, matrigel, an extracellular matrix (ECM), ECM analogs, laminin, fibronectin, or collagen. Those of skill in the art however will recognize that this is a non-limiting list and that other molecules can be used to attach cells to a solid surface. Methods for initial attachment of the tethers to the solid surface are known in the art.

The skilled person will be familiar with the culture medium composition and culture conditions for:
promoting reprogramming of a cell towards a pluripotent state;
promoting a naïve pluripotent state; and
promoting a primed pluripotent state.

In one embodiment, the medium for promoting a hypomethylated DNA state, such as the naïve pluripotent state comprises a MEK inhibitor, a PKC inhibitor, a GSK3 inhibitor, a STAT3 activator and a ROCK inhibitor.

Reference to a MEK inhibitor refers to MEK inhibitors in general. A MEK inhibitor may inhibit any member of the MEK family of protein kinases, including MEK1, MEK2, and MEK5. Examples of suitable MEK inhibitors are known in the art and include PD184352 and PD98059, inhibitors of MEK1 and MEK2 U0126 and SL327. In particular, PD184352 and PD0325901 have been found to have a high degree of specificity and potency compared to other known MEK inhibitors.

Examples of protein kinase C (PKC) inhibitors include, Gö6983 (3-[1-[3-(dimethylamino)propyl]-5-methoxy-1H-indol-3-yl]-4-(1H-indol-3-yl)-1H-pyrrole-2,5-dione (Gschwendt et al., 1996 FEBS Lett 392:77-80). Another preferred PKC inhibitor is Ro-31-8425. Preferably the PKC inhibitor is present in the medium at a concentration of between 0.01 to 10 μM, 0.1 to 5 μM, preferably 1 to 4 μM.

Reference to GSK3 inhibition refers to inhibition of one or more GSK3 enzymes. The family of GSK3 enzymes is well known and a number of variants have been described. In certain embodiments, GSK3β is inhibited. GSK3-α inhibitors may also be used. A wide range of GSK3 inhibitors is known, by way of examples, the inhibitors CHIR 98014, CHIR 99021, AR-A0144-18, TZD-8, SB21676763 and SB415286.

Inhibitor may be provided or obtained by those skilled in the art by convention means or from conventional sources. The inhibitor may be a small molecule inhibitor or an interfering RNA (RNAi). The skilled person will also be familiar with various methods and assays for identifying kinase inhibitors.

Examples of STAT3 activators include LIF, preferably hLIF.

The combination of a MEK inhibitor, a GSK3 inhibitor and LIF may be referred to as 2iL.

As used herein, a ROCK inhibitor refers to an inhibitor of Rho-binding kinase. Examples of such inhibitors include ((1R,4r)-4-((R)-1-aminoethyl)-N-(pyridin-4-yl)cyclohexane carboxamide, Abcam), also known as trans-N-(4-(1-aminoethyl)-cyclohexanecarboxamide, 1-(5-isoquinolunyl) (sulfonyl) homopiperazine (1-(5-isoquinolinylsulfonyl)homopiperazine. Typically the amount of ROCK inhibitor will be between about 0.1 to 50 μM, preferably about 1 to 10 μM.

“Inhibitors,” “activators,” and “modulators” of expression or of activity are used to refer to inhibitory, activating, or modulating molecules, respectively, identified using in vitro and in vivo assays for expression or activity of a described target protein, e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100% Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

As used herein, “inhibit,” “prevent” or “reduce,” or “inhibiting,” “preventing” or “reducing” are used interchangeably herein. These terms refer to the decrease in a measured parameter (e.g., activity, expression, mitochondrial respiration, mitochondrial oxidation, oxidative phosphorylation) in a treated cell (tissue or subject) in comparison to an untreated cell (tissue or subject). A comparison can also be made of the same cell or tissue or subject between before and after treatment. The decrease is sufficient to be detectable. In some embodiments, the decrease in the treated cell is at least about 10%, 20%, 30%, 40%, 50%, 60%, 70%, 80%, 90%, or completely inhibited in comparison to an untreated cell. In some embodiments the measured parameter is undetectable (i.e., completely inhibited) in the treated cell in comparison to the untreated cell.

Examples of suitable culture medium are summarised in Table 2 below:

TABLE 2
Cell culture media that can be used to culture different cell types

| Cell | Media | Cat#: | Company/Reference |
|---|---|---|---|
| Fibroblasts | Medium 106 | N/A | Life Technologies |
| | DMEM (high | | |
| | glucose, without | | |
| | sodium pyruvate), | | |
| | 10% FBS, 1% | | |
| | penicillin/streptomycin, | | |
| | 1% nonessential | | |
| | amino acids, 1 mM | | |
| | Glutamax, 0.1 mM | | |
| | 2-mercaptoethanol | | |
| | and 1 mM sodium | | |
| | pyruvate | | |
| | Essential 6 | #A1516401 | ThermoFisher |
| | Essential 8 | #A1517001 | ThermoFisher |
| Hypomethylation/ | T2iLGoY | N/A | Guo et al., (2016) Stem Cell |
| Naïve media | | | Reports, 6(4): P437-446 |
| | 5iLAF | N/A | Theunissen et al., (2014) Cell Stem |
| | | | Cell, 15: 471-487 |
| | RSeT | #05978 | StemCell Technologies |
| | PXGL | N/A | Bredenkamp et al., (2019) Stem Cell |
| | | | Reports, |
| | | | https://doi.org/10.1016/j.stemcr.2019.10.009 |
| | NHSM | N/A | Gafi et al., (2013) Nature, |
| | | | 504(7479): 282-6 |
| | hPGCLC Induction | N/A | Irie et al (2015) Cell, 160: 253-268 |
| | medium | | |
| Primed | KSR/FGF2 | N/A | Eiselleovat et al., (2009) Stem Cells, |
| media | | | 27: 1847-57 |
| | Essential 8 | #A1517001 | ThermoFisher |
| | mTeSR | #85850 | StemCell Technologies |
| | AKIT | N/A | Yasuda et al., (2018) Nature |
| | | | Biomedical Engineering, 2: 173-182 |
| | B8 | N/A | Kuo et al., (2019) |
| | | | http://dx.doi.org/10.1101/685503. |

As used herein, hPGCLC induction medium refers to human primordial germ cell-like cells induction medium. It will be understood that this medium can be used to promote a state of global hypomethylation in the cells.

RSeT™ Medium is a defined cell culture medium used for the maintenance of naïve-like hPSCs under feeder-dependent and hypoxic conditions. RSeT™ Medium contains pre-screened quality components and does not contain bFGF or TGFβ. It is compatible with human embryonic stem (ES) cells and human induced pluripotent stem (iPS) cells.

hPSCs cultured in RSeT™ Medium exhibit features of a naïve-like state such as tightly packed, domed colonies with refractive edges. Key transcripts associated with naïve-like hPSCs such as KLF2, KLF4, and TFCP2L1 show increased expression in hPSCs cultured in RSeT™ Medium. RSeT™ hPSCs can be converted back to a primed state by culture in mTeSR™ 1 and can then be differentiated using standard techniques.

Exemplary t2iLGoY medium:
a 50:50 mixture of DMEM/F-12 (Gibco) and Neurobasal medium (Gibco), supplemented with 2 mM L-glutamine (Gibco),
0.1 mM 2-mercaptoethanol (Gibco),
0.5% N2 supplement (Gibco),
1% B27 supplement (Gibco),
1% Pen-strep (Gibco),
10 ng/ml human LIF (made in house),
250 μM L-ascorbic acid (Sigma),
10 μg/ml recombinant human insulin (Sigma),
1 μM PD0325901 (Miltenyi Biotec),
1 μM CHIR99021 (Miltenyi Biotec),
2.5 μM Gö6983 (Tocris),
10 μM Y-27632 (abcam).

Differentiation

The present invention also includes a method of generating differentiated cells from the iPSCs made according to the present invention.

The differentiated cells may be used to create cell culture models of human development and disease that can be applied in drug discovery and development, and in teratogenicity and toxicology testing; source of tissue stem cells and more mature cells for applications in clinical cell therapy; analysis of the relative contributions of genetics and epigenetics to developmental disorders, genetic disease and quantitative traits to facilitate advances in diagnostics, prognostics and patient treatment; generation of tissues and organs for transplantation either by bioengineering in vitro or by lineage/organ specific contribution to human-animal chimaeras.

The iPSCs made according to the present invention may be differentiated into any somatic cell, including but not limited to: neurons, astrocytes, oligodendrocytes, keratinocytes, epithelial cells, cardiomyocytes, fibroblasts, B cells, T cells, macrophages, haematopoietic stem cells, retinal epithelial cells, smooth muscle cells, skeletal cells, podocytes, renal cells, hepatocytes, pancreatic β-islets, lung alveolar cells, adipocytes, chondrocytes and osteocytes.

Further still, the iPSCs made according to the present invention may be differentiated into primordial germ cells (PGCs) and gametes.

The skilled person will be familiar with standard methods for differentiating the iPSCs generated according to the present invention. There is an abundance of literature available to the skilled person providing instructions on differentiation of iPSCs. Kits including differentiation media for the differentiation of iPSCs to somatic cell types are also commercially available including from Sigma Aldrich and ThermoFisher Scientific.

Successful differentiation to a somatic cell type can be determined by determining the presence of one or more markers that are characteristic of the somatic cell. Further, successful differentiation can be determined by observing for a change in morphology from pluripotent state to differentiated morphology, characteristic of the target cell type.

The differentiated cell, or population of differentiated cells obtained or obtainable according to the present methods can be characterised by cells having fewer characteristics of the somatic cell (i.e., the somatic cell subjected to step a) of the invention) as compared to a differentiated cell, or population of differentiated cells obtained by differentiation of an iPSC produced according to conventional methods (i.e., without transient exposure to the second culture medium or second culture conditions as described herein). Further, the population of differentiated cells may have fewer cells having characteristics of the somatic cells.

More specifically, and as further demonstrated herein in the Examples, populations of cells differentiated from primed iPSCs obtained according to the methods of the prior art, typically include a subset of cells which retain characteristics of the source cell. In other words, if reprogramming from a fibroblast, any differentiated cells obtained from iPSCs generated from the fibroblast when reprogramming in primed media, typically include some cells which resemble fibroblasts, both in morphology and gene expression characteristics. The iPSCs produced according to the present invention have an epigenetic profile that reduces the likelihood of differentiated cells having characteristics of the original source cell used in reprogramming.

Pharmaceutical Compositions and Other Applications

The invention, according to certain embodiments, contemplates the use of any cells, tissues and/or organs/organoids generated using the iPSCs generated according to the invention.

The isolated cells of the present invention may be further used for disease modelling, drug screening and patient-specific cell based therapy.

Thus, according to a certain aspect of the invention, there is provided an isolated iPSC produced according to the methods of the invention or an isolated differentiated cell obtained from an iPSC obtained or obtainable according to the methods of the invention.

Introducing cells may be performed in vitro or ex vivo via direct injection into the subject requiring treatment.

As used herein the phrase “subject in need thereof” refers to a mammalian subject (e.g., human being) who is diagnosed with the pathology. In a specific embodiment, this term encompasses individuals who are at risk to develop the pathology. Veterinary uses are also contemplated. The subject may be of any gender or at any age including neonatal, infant, juvenile, adolescent, adult and elderly adult.

The iPSCs obtained or obtainable according to the present invention, or differentiated cells obtained therefrom, may be transplanted to a subject per se, or in a pharmaceutical composition where they are mixed with suitable carriers or excipients. Similarly, the constructs of the present invention may be administered to a subject per se, or in a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cells of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

Typically, the pharmaceutical composition is administered in a local rather than systemic manner, for example, via injection of the pharmaceutical composition directly into a tissue region of a patient.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Pharmaceutical compositions suitable for use in context of the present invention include compositions wherein the active ingredients are contained in an amount effective to achieve the intended purpose. Determination of a therapeutically effective amount is well within the capability of those skilled in the art, especially in light of the detailed disclosure provided herein.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser advice may be a syringe. The syringe may be prepacked with the cells. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labelling approved by the U.S. Food and Drug Administration for prescription drugs or of an approved product insert. Compositions comprising a preparation of the invention formulated in a compatible pharmaceutical carrier may also be prepared, placed in an appropriate container, and labelled for treatment of an indicated condition, as if further detailed above.

The present invention includes the following non-limiting Examples.

### EXAMPLES

### Example 1: Reprogramming of Somatic Cells to iPSCs (“Transient Naïve Treatment” Protocol)

Primary adult human dermal fibroblasts (HDFa, Life Technologies) were expanded in medium 106 (Life Technologies) supplemented with LSGS (Gibco) for nuclear reprogramming experiments.

Fibroblast cells at an early passage stage (<P6) were seeded into 6-well plates at 50,000-70,000 cells per well before transduction in fibroblast medium containing DMEM (Gibco), 10% FBS (Hyclone), 1% nonessential amino acids (Gibco), 1 mM GlutaMAX (Gibco), 1% Pen-strep (Gibco), 0.1 mM 2-mercaptoethanol (Gibco) and 1 mM sodium pyruvate (Gibco).

48 hours later, cells in one well were trypsinized for counting to determine the volume of viruses required for transduction (MOI). Transduction was performed using CytoTune 2.0 iPSC Sendai Reprogramming Kit (Invitrogen), consisting of four transcription factors, OCT4, SOX2, cMYC, KLF4.

24 hours later, the viruses were removed and media changes were performed every other day. 7 days post transduction, cells were harvested using TrypLE express (Life tech) and re-seeded onto a layer of irradiated MEF feeders in fibroblast media, then the following day, media were switched to naïve medium (2iLGoY, Guo et al., see Table 2).

5 days later when dome-shaped colonies were evident, intermediate cells were harvested using Accutase (Stem Cell Technologies) and reseeded onto vitronectin (Life tech)-coated plates in naïve conditions. The following day, the medium was switched to primed medium (Essential 8 (Life tech)). Alternatively, media switching from naive to primed can be performed without splitting the cells. When the culture became confluent, cells were harvested using a 0.5 mM EDTA/PBS solution (Invitrogen), and maintained in vitronectin/E8 feeder free human pluripotent stem cell culture system. Cells were cultured in a 37° C., 5% O₂ and 5% CO₂ incubator and media changed on a daily basis. Cells were passaged every 4-5 days.

### Example 2: Correction of Epigenome in Established iPSCs (“Naïve-to-Primed Protocol”)

Primary adult human dermal fibroblasts (HDFa, Life Technologies) were expanded in medium 106 (Life Technologies) supplemented with LSGS (Gibco) for nuclear reprogramming experiments.

Fibroblast cells at an early passage stage (<P6) were seeded into 6-well plates at 50,000-70,000 cells per well before transduction in fibroblast medium containing DMEM (Gibco), 10% FBS (Hyclone), 1% nonessential amino acids (Gibco), 1 mM GlutaMAX (Gibco), 1% Pen-strep (Gibco), 0.1 mM 2-mercaptoethanol (Gibco) and 1 mM sodium pyruvate (Gibco).

48 hours later, cells in one well were trypsinized for counting to determine the volume of viruses required for transduction (MOI). Transduction was performed using CytoTune 2.0 iPSC Sendai Reprogramming Kit (Invitrogen), consisting of four transcription factors, OCT4, SOX2, cMYC, KLF4.

24 hours later, the viruses were removed and media changes were performed every other day. 7 days post transduction, cells were harvested using TrypLE express (Life tech) and re-seeded onto a layer of irradiated MEF feeders in fibroblast media, then the following day, media were switched to naïve medium (2iLGoY).

16-18 days post-transduction (i.e., after 8-10 days in naïve condition), naïve iPSCs were harvested using Accutase (Stem cell technologies) and passaged more than 10 times to obtain established naïve iPSCs.

Naïve phenotype for the established naïve iPSCs was confirmed by flow cytometry, immunostaining for naïve pluripotency-associated markers.

Naïve iPSCs were then harvested using Accutase (Stem Cell Technologies) and reseeded onto vitronectin (Life tech)-coated plates in naïve condition. The medium was switched to Essential 8 (Life tech) medium the following day. When the culture became confluent, cells were harvested using a 0.5 mM EDTA/PBS solution (Invitrogen), and maintained in vitronectin/E8 feeder-free human pluripotent stem cell culture system. Cells were cultured in a 37° C., 5% O₂ and 5% CO₂.

### Example 3: Epigenetic Profiling of iPSCs at Different Stages of Reprogramming

Acquisition of Aberrant DMRs During Reprogramming

1. Whole Genome Bisulfite Sequencing (by MethylC-Seq) and DNA Methylation Analyses

DNA methylation analysis was performed using FACS isolated cells. Genomic DNA was isolated with the DNeasy Blood and Tissue Kit according to manufacturer's instructions. Libraries were prepared using genomic DNA with 0.5% (w/w) unmethylated lambda phage DNA as described in (Urich et al. (2015) Nature Protocols 10:3 p 475-483), fragmented with a Covaris S2 sonicator to a mean length of 200 bp. Fragments were end-repaired, A-tailed, and ligated to methylated Illumina TruSeq adapters or B100 Nextflex adapters followed by bisulfite conversion using EZ DNA-methylation Gold kit (Zymo Research). Library fragments were then subjected to 4 to 9 cycles of PCR amplification with KAPA HiFi Uracil+ DNA polymerase (KAPA Biosystems). Sequencing was performed on Illumina HiSeq 1500 or NovaSeq 6000. Sequencing adapters were trimmed with BBduk with the options mink=3, qtrim=r, trimq=10 minlength=20, before alignment to hg19 with Bowtie2 and BSseeker2 with the option—n 1 (Chen et al. (2010) BMC Bioinformatics 11 p 203; Langmead et al. (2009) Genome Biology 10:3 R25). PCR duplicates were removed using Sambamba before methylation quantification with BSseeker2 (Chen et al. 2010; Tarasov et al. (2015) Bioinformatics 31:2 p 2032-3024). Read counts for CG dinucleotides were aggregated for both strands before downstream analyses. Differentially methylated regions (DMRs) were statistically determined using the DMRseq algorithm (Korthauer et al. (2018) Biostatistics 20:3 p 367-383) with DMRs considered significant if the methylation difference was greater than 20% and a DMR P-value <0.05. PMDs were determined using fibroblast methylation profiles and a hidden markov model as implemented in MethylSeekR (Burger et al. (2013) Nucleic Acids Research 41:16 e155). All non-CG DNA methylation values were corrected for the non-conversion rate determined by the spiked lambda phage DNA methylation levels.

Acquisition of aberrant DMRs during reprogramming was identified by first calling DMRs between primed iPSC lines and ESC lines. This included 4 ESC replicates (32F passage 3 in KSR media; 32F passage 10 in KSR media; 32F passage 26 in E8 media; 38F passage 20 in E8 media) and 5 ESC replicates (H1 in mTeSR1 media; HESO7 in KSR media; HESO8 in KSR media; H9 in mTeSR1 media; H9 in E8 media). Subsequently, DMRs were also called between primed iPSC lines and the progenitor fibroblast cells (2 replicates; 1×32F fibroblast and 1×38F fibroblast). DMRs were classified into two classes. The first class was designated as ‘memory DMRs’ and were those regions determined to be significantly different between iPSCs and ESCs and which did not overlap any DMR called between fibroblasts and iPSCs. In these regions, the methylation levels in iPSCs was more similar to fibroblasts than ESCs. The second class of DMRs was designated as ‘iPSC DMRs’ and were those regions determined to be significantly different between iPSCs and ESCs and which also overlap a DMR determined to be significantly different between iPSCs and fibroblasts. In these regions, the methylation level in iPSCs was distinct from both ESC and the progenitor fibroblasts.

Naïve-to-Primed Reprogramming Erases Somatic Cell Memory and Produces Cells More Closely Resembling ESCs

With respect to DNA methylation, a DMR in a naive-to-primed iPSC or TNT-primed iPSC more closely resembles ESCs when the average methylation level in the regions is less than 20% compared to ESCs lines. With respect to gene and transposable element expression, the difference between naive-to-primed cells and ESC lines is less than 2-fold.

RNA-Sequencing (RNA-Seq)

RNA extraction was performed using the RNeasy micro kit (Qiagen, Cat #74004) from −2-20×10⁴ cells with QIAcube (Qiagen). The concentrations of RNA were measured by a Qubit RNA HS Assay Kit (ThermoFisher, Cat #Q32855) on a Qubit 2.0 Fluorometer (ThermoFisher). ˜25 ng of RNA was used for library construction with the SPIA kit (NuGen) follow the manufacturer's instructions and subsequently sequenced by HiSeq 1500 or HiSeq 3000 sequencer (Illumina). Sequencing libraries were sequenced in single end format with 50 nt length reads and a targeted number of reads of 20-30 M. RNA-seq reads were aligned with HISAT2 to hg19 (Kim et al., (2015) Nature Methods 12:4 357-360), with gene and transposable element quantification performed with TEtranscripts (Jin et al., (2015) Bioinformatics 31:22 p 3593-3599) using the recommended parameters for the human genome. Differential expression of genes and transposable elements was calculated using edgeR (Robinson et al., (2010) Bioinformatics 26:1 139-140) with TMM normalisation. Differential expression was defined as genes or transposable elements with >2-fold expression difference and a false discovery rate <0.01.

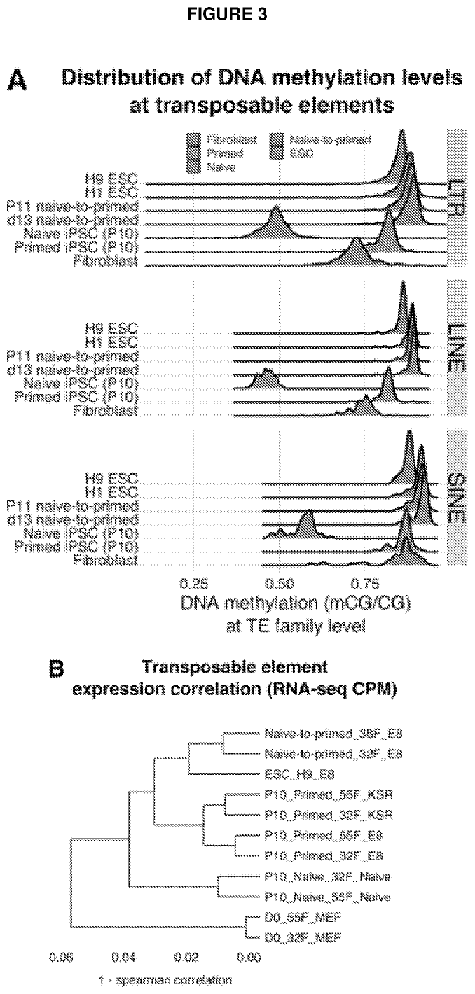

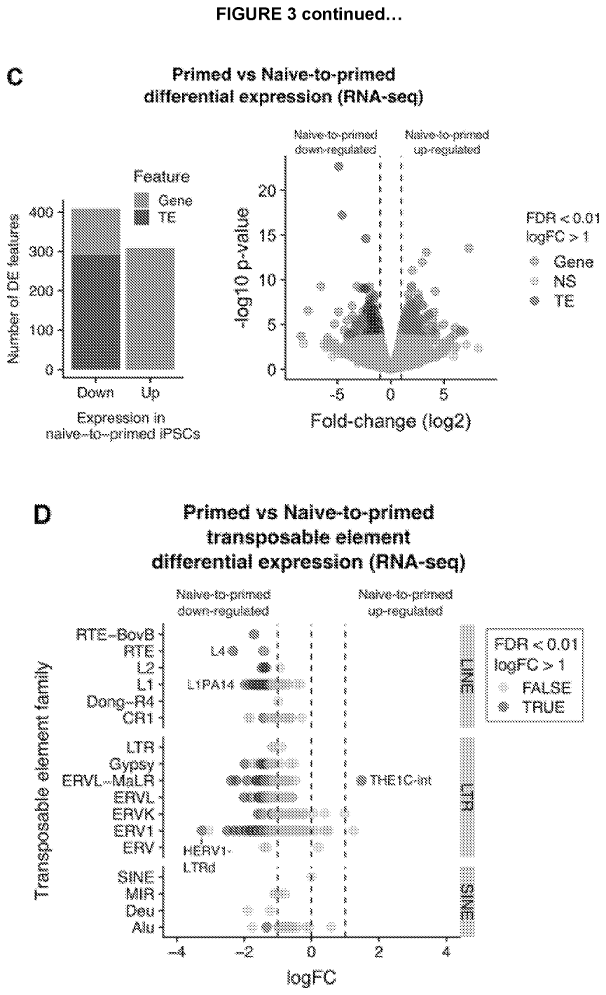

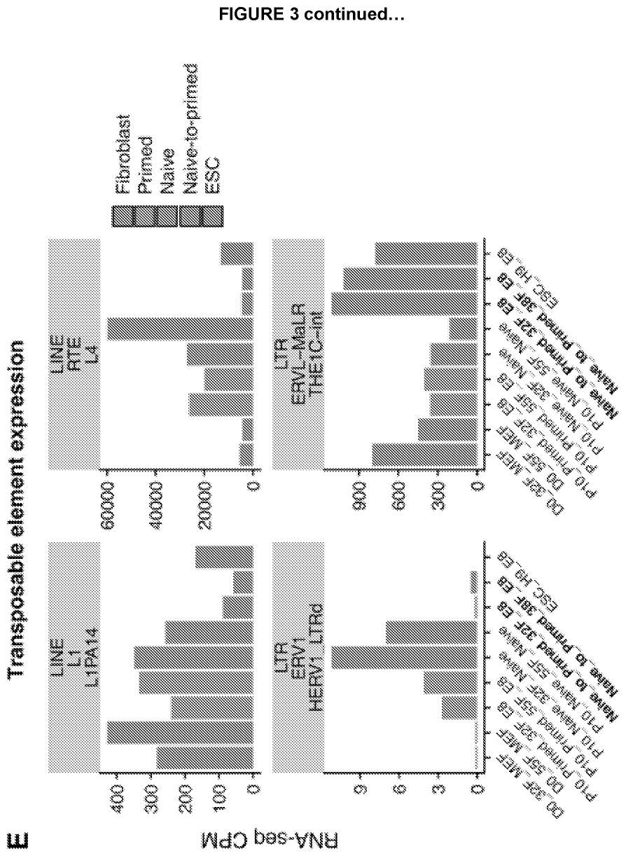

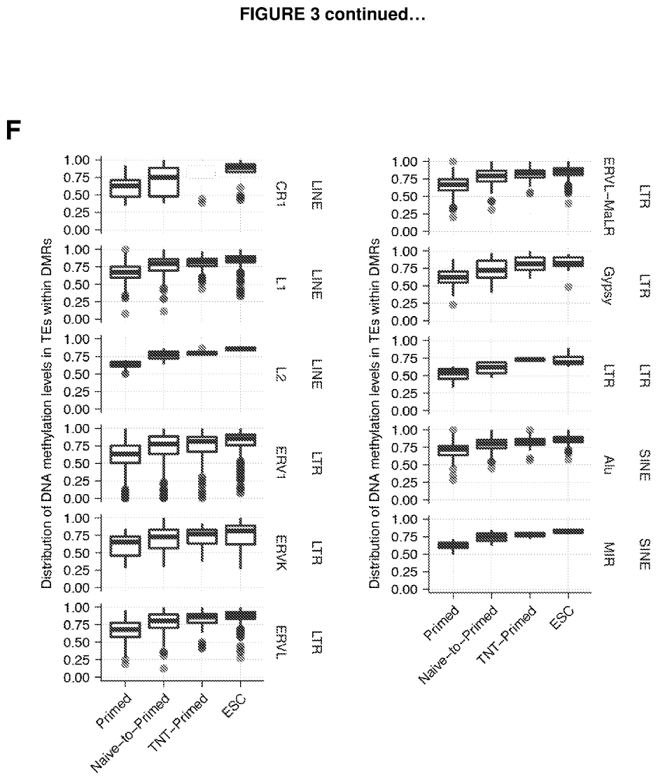

FIG. 3A shows histograms of the DNA methylation levels at TEs in naïve-to-primed cells compared to ESCs and iPSCs produced according to conventional methods. FIG. 3B shows dendrograms depicting that TE expression indicating naïve-to-primed iPSCs more closely resemble ESCs than primed iPSCs.

Differentiation Scorecard Assessment

Embryoid body differentiation was performed following manufacturer's instructions of the ScoreCard assay (Invitrogen). Briefly, pluripotent cells were dissociated into cell clumps and then transferred into a nonadherent dish with EB differentiation medium. EBs were harvested after 7 d and lysed, and total RNA was extracted with the QIAcube (Qiagen)/RNeasy micro kit (Qiagen) and reverse transcribed using the SuperScript III cDNA synthesis kit (Invitrogen). cDNA was then added into 384-well ScoreCard plates (Invitrogen) together with TaqMan Gene Expression Master Mix (Thermo Fisher), and RT-PCR was carried out with the 7900HT Real-Time PCR System (Thermo Fisher). The resulting data were analyzed using hPSCs ScoreCard analysis software (Thermo Fisher).

The data shown in FIG. 4 depicting the differentiation scorecard for embryoid bodies derived from primed and naïve-to-primed iPSCs, shows that naïve-to-primed iPSCs exhibit less differentiation bias towards the mesoderm lineage compared to primed iPSCs (fibroblasts being of mesoderm origin and being the somatic cell type prior to reprogramming to pluripotency).

### Example 4: Differentiation of Naïve-to-Primed iPSCs

Cortical Neuron Differentiation from MEL1-hESC-Fibroblasts-Derived iPSCs

Two days prior to differentiation (day 2), hiPSCs were plated on matrigel (BD Biosciences) coated culture vessels at low density (0.5-1×10⁴ cells/cm²) in E8 medium supplemented with 10 μM Y-27632 (Sigma-Aldrich). On day 0, E8 medium was changed to DDM medium (DMEM/F12+GlutaMax supplemented with 1× N2 supplement (Life Technologies), 0.1 mM non-essential amino acids (Life Technologies), 1 mM sodium pyruvate (Life Technologies), 500 μg/ml bovine serum albumin (Life Technologies), 0.1 mM 2-mercaptoethanol (Life Technologies) and 50 W/ml Pen/Strep) (Life Technologies) supplemented with 2% B27 (without vitamin A) (Life Technologies) and 100 ng/ml LDN (Stem Cell Technologies), and replenished every second day. The percentage of NCAM (Miltenyi Biotec) positive cells was assessed on Day 8 and 16 by flow cytometry analysis.

Neural Stem Cell (NSC) Generation from iPSCs

iPSCs were cultivated in E8 media (Life Technologies) on Cultrex (R&D Systems) coated TC dishes and split 1:10 every 5 days. Colonies were mechanically disaggregated with 0.5 mM EDTA in PBS (Sigma). 10 μM ROCK Inhibitor (Selleckchem) was added for 24 hours after splitting.

After splitting, colonies were collected by sedimentation and resuspended in E8 media with ROCK inhibitor and cultured in petri dishes to form embryoid bodies (EBs) in suspension. After 24 h, media was changed to Knockout DMEM (Life Technologies) with 20% Knockout Serum Replacement (Life Technologies), 1 mM beta-mercaptoethanol (Sigma), 1% nonessential amino acids (NEAA, Life Technologies), 1% penicillin/streptomycin (Life Technologies) and 1% Glutamax (Life Technologies) supplemented with 10 μM SB-431542 (Selleckchem), 1 μM dorsomorphin (Selleckchem) for neural induction, as well as 3 μM CHIR 99021 (Cayman Chemical) and 0.5 μM PMA (Sigma). Medium was replaced on day 3 by N2B27 medium (50% DMEM-F12 (Life Technologies), 50% Neurobasal (Life Technologies) with 1:200 N2 supplement (R&D Systems), 1:100 B27 supplement lacking vitamin A (Miltenyi Biotec) with 1% penicillin/streptomycin (Life Technologies) and 1% Glutamax (Life Technologies) supplemented with the same small molecule supplements. On day 4, SB-431542 and dorsomorphin were withdrawn and 150 μM Ascorbic Acid (AA; Sigma) was added to the medium. On day 6, the EBs were triturated with a 1,000 μL pipette into smaller pieces and plated on Cultrex-coated 12-well plates at a density of about 10-15 per well in NSC expansion medium (N2B27 with CHIR, PMA, and AA). After another 5 days, cells were split at a ratio of 1:5 using Trypsin-EDTA (Life Technologies) and Trypsin inhibitor (Sigma) onto a new Cultrex-coated well. After another 5 days, cells were collected by 10 min trypsinization at 37 C to generate a single cell suspension for the 10×3′ single cell RNA-seq v3 workflow (10× Genomics).

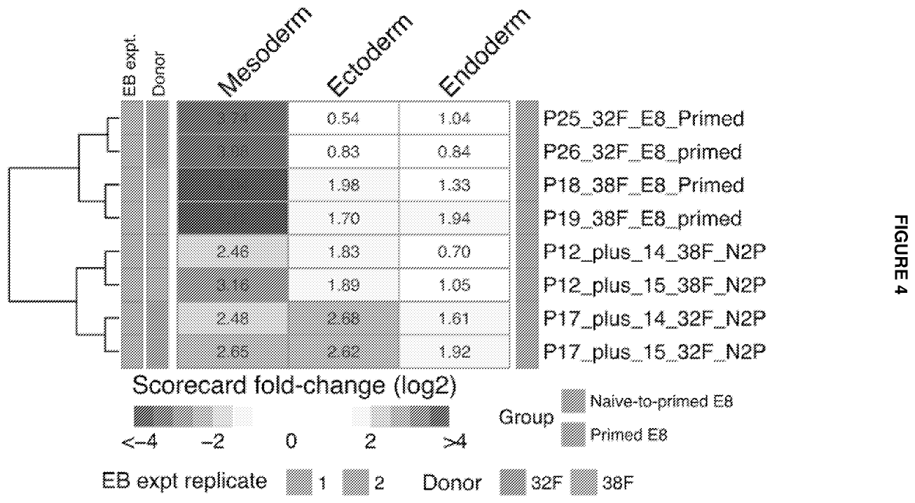

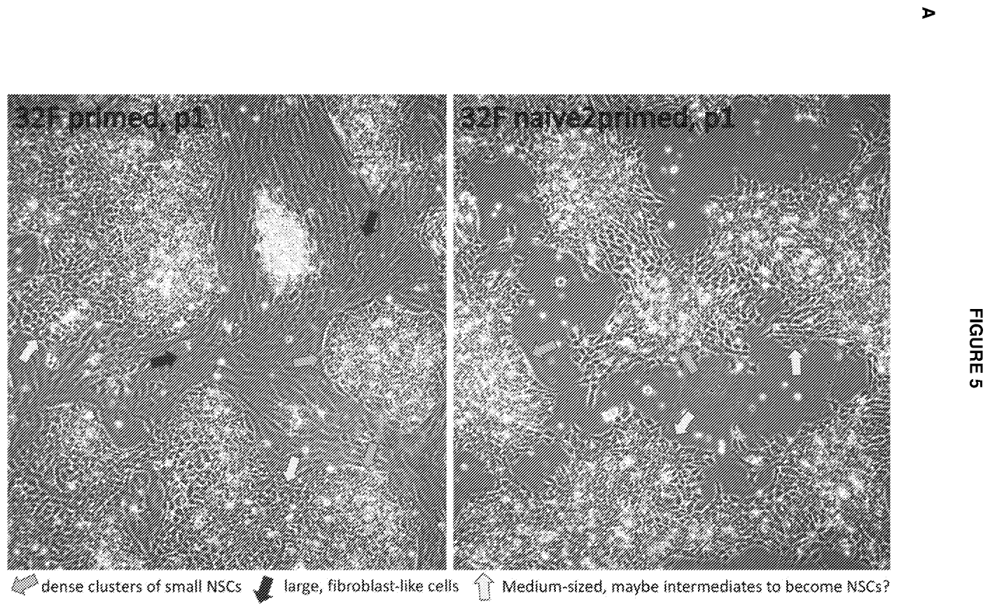

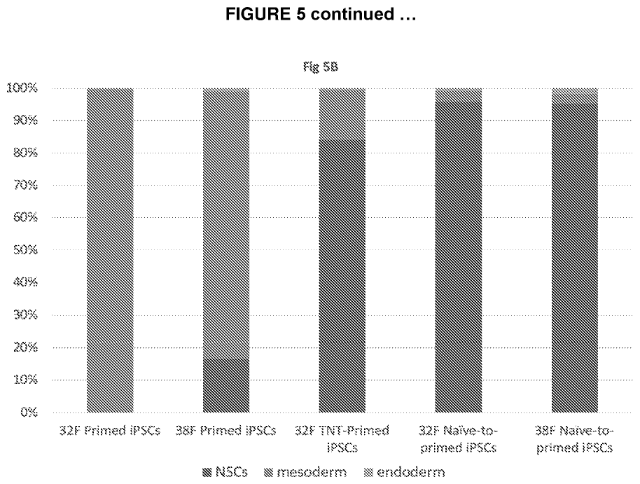

FIG. 5A shows images of NSCs from primed iPSCs and naïve-to-primed iPSCs. These images show that the differentiation of primed iPSCs to NSCs produces fibroblast-like cells in the spaces between NSC colonies. The presence of the fibroblast-like cells is significantly reduced when differentiating naïve-to-primed iPSCs to NSCs.

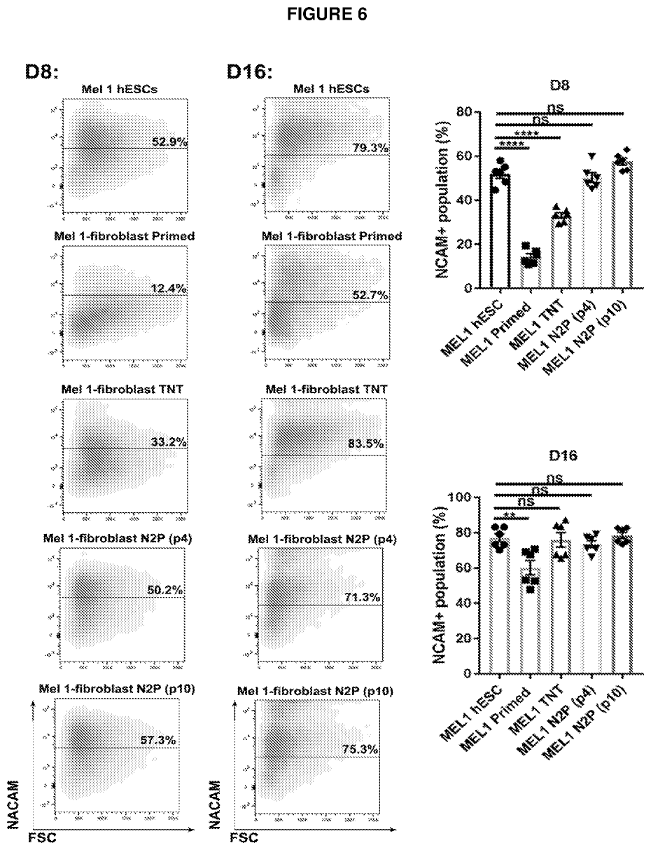

FIG. 6 shows the results of flow cytometry of MEL1-derived iPSCs produced according to the present invention (naïve-to-primed and TNT-primed), MEL1-derived hESCs and MEL1-derived iPSCs produced from primed iPSCs. The results show that the percentage of NCAM positive cells on day 8 and day 16 of cortical neuronal differentiation from TNT-primed and naïve-to-primed iPSCs more closely resembles the percentage obtained from ESCs than from primed iPSCs.

Single Cell RNA-Seq and Hash Tagging

Single cell suspensions were counted using a haemocytometer and 200,000 cells per sample used for incubation with hashtag antibodies. Cells were filtered through a 40 μm cell strainer, centrifuged at 800 g for 5 min and resuspended in a total volume of 46 μl cell staining buffer (2% BSA (Sigma), 0.01% Tween (Sigma) in 1×DPBS (Life Technologies) with 4 μl of Fc blocking reagent (Biolegend) and incubated for 10 min on ice. Then, each sample received 0.2 μg of a different TotalSeq-A anti-human Hashtag antibody (Biolegend) and were incubated for 30 min on ice for antibody binding. After the incubation, 1 ml of cell staining buffer was added, and sample centrifuged at 300 g for 3 min. Supernatant was removed and wash repeated for a total of 3 washes to remove all unbound antibodies. Cells were counted again, and equal cell numbers for of each sample combined to get a cell concentration suitable for loading on the 10× Chromium controller aiming to get 10,000 cells represented. The mixed cell suspension was filtered one more time using a 40 μm cell strainer and processed for single cell RNA-seq on 10×3′ v3 chemistry following the manufacturer's instructions.

Libraries for single cell RNAseq were made following the standard workflow, while HTO libraries for hashtag information were generated as follows: during the cDNA amplification step, HTO primers were added to allow amplification of the HTO barcodes, and supernatant from the first step of clean-up after cDNA amplification PCR was not discarded but used to prepare the HTO library. HTO products were purified using 2× SPRI and amplified for 8 PCR cycles with 10× SI-PCR oligo and TruSeq Small RNA RPIx primers to generate a library of around 180 bp size.

Single Cell RNA-Seq (scRNA-Seq) Analysis

Sequencing was performed on a NovaSeq 6000 sequencer to generate around 420 million reads for the single cell RNA-seq library and around 40 million reads for the HTO library. RNA-seq fastq files were processed using CellRanger count 3.1.0, while HTO fastq files were processed using CITE-seq-Count 1.4.3. Both datasets were loaded into Seurat 3.1 and combined by intersecting them based on cell barcodes. RNA data was log normalized, variable features detected by mean variance while HTO data was normalized by centred log-ratio transformation. HTODemux was used to assign single cells back to their sample origins and to exclude doublets and negatives from further analysis. Top 1000 most variable features were used for scaling and PCA of RNA data, using 10 dimensions with a resolution of 0.6 for clustering and tSNE.

Cluster identities were defined based on the expression of markers for mesoderm (BMP1, BMP4, HAND1, SNAI1, TGFB1, TGFB2), endoderm (CLDN6, FOXA1, NODAL), neural stem cells (NES, RBFOX3, PAX3, PAX6, SOX1, SOX2) or mitochondria (low UMI counts and mitochondrial transcripts enriched). Using the HTO identity for each cell, the proportion of cell identities within each of the samples used could be defined

The data shown in FIG. 5B shows that both naïve-to-primed and TNT-primed cells show substantially fewer fibroblast-like cells as characterised by marker gene expression.

It will be understood that the invention disclosed and defined in this specification extends to all alternative combinations of two or more of the individual features mentioned or evident from the text or drawings. All of these different combinations constitute various alternative aspects of the invention.

TABLE 3
Summarised CG DNA methylation level values (0 to 1) in corrected CG-DMRs for ESC,
Primed-iPSCs (Primed), Naive-to-Primed iPSCs (NtoP), TNT-Primed iPSCs (TNT), and Fibroblast cells.

| Chromosome: | | | | | | TNT | NtoP |
|---|---|---|---|---|---|---|---|
| coordinate range | ESC | Primed | TNT | NtoP | Fibroblast | corrected | corrected |
| chr8:138623197-138624595 | 0.86934897 | 0.62373709 | 0.82043486 | 0.85191059 | 0.52452389 | TRUE | TRUE |
| chr8:138643882-138644739 | 0.91256105 | 0.69422569 | 0.90674263 | 0.93317509 | 0.62679739 | TRUE | TRUE |
| chr8:138667985-138670410 | 0.90234265 | 0.67582567 | 0.8670826 | 0.87515803 | 0.58998475 | TRUE | TRUE |
| chr8:138694062-138694511 | 0.89665912 | 0.59420868 | 0.8672117 | 0.85366126 | 0.58233652 | TRUE | TRUE |
| chr8:138707838-138708702 | 0.89445225 | 0.59833133 | 0.85938356 | 0.89503508 | 0.51358696 | TRUE | TRUE |
| chr8:138731564-138732232 | 0.89209463 | 0.63716422 | 0.86503136 | 0.86431175 | 0.56894944 | TRUE | TRUE |
| chr8:138756711-138758160 | 0.89251983 | 0.65326261 | 0.87843923 | 0.88554513 | 0.56092131 | TRUE | TRUE |
| chr8:138837216-138837924 | 0.91215968 | 0.67779287 | 0.89675325 | 0.92840249 | 0.64356061 | TRUE | TRUE |
| chr8:138875382-138876050 | 0.89420338 | 0.61836366 | 0.90441943 | 0.85897869 | 0.61564626 | TRUE | TRUE |
| chr8:138931545-138932964 | 0.84838963 | 0.64308545 | 0.82057207 | 0.80102973 | 0.54740152 | TRUE | TRUE |
| chr8:138973055-138974148 | 0.89392949 | 0.67409299 | 0.90518274 | 0.87458032 | 0.61072777 | TRUE | TRUE |
| chr8:139026522-139027107 | 0.91423872 | 0.70001082 | 0.89909256 | 0.90263987 | 0.60196399 | TRUE | TRUE |
| chr8:139067878-139068345 | 0.91577495 | 0.62153114 | 0.87084054 | 0.88016325 | 0.54302059 | TRUE | TRUE |
| chr8:139200097-139201035 | 0.85430494 | 0.61132004 | 0.89478969 | 0.82815114 | 0.62570383 | TRUE | TRUE |
| chr8:139320938-139324378 | 0.85226321 | 0.62332036 | 0.8597555 | 0.80891802 | 0.52687627 | TRUE | TRUE |
| chr8:139379244-139381039 | 0.92379216 | 0.6924601 | 0.90666796 | 0.87638452 | 0.61291468 | TRUE | TRUE |
| chr8:139536006-139536239 | 0.89065862 | 0.65299812 | 0.8771361 | 0.86802365 | 0.5764557 | TRUE | TRUE |
| chr8:139650925-139652435 | 0.88310604 | 0.64777187 | 0.84014248 | 0.8401501 | 0.57664742 | TRUE | TRUE |
| chr7:153936245-153937376 | 0.92319834 | 0.68362842 | 0.94339769 | 0.88715886 | 0.58457592 | TRUE | TRUE |
| chr7:154009348-154009801 | 0.91742977 | 0.71152355 | 0.91873983 | 0.88329903 | 0.63033529 | TRUE | TRUE |
| chr7:154246590-154246927 | 0.87927267 | 0.6716887 | 0.88950834 | 0.84553241 | 0.68442728 | TRUE | TRUE |
| chr7:154385686-154386451 | 0.90424304 | 0.66398123 | 0.87638288 | 0.8556276 | 0.60691708 | TRUE | TRUE |
| chr16:6608145-6608875 | 0.07627737 | 0.28637019 | 0.05085307 | 0.06653889 | 0.31990344 | TRUE | TRUE |
| chr16:6819104-6819935 | 0.8806054 | 0.59793475 | 0.86256637 | 0.88372064 | 0.58553114 | TRUE | TRUE |
| chr16:6831151-6831843 | 0.88943553 | 0.66766087 | 0.84395654 | 0.90017909 | 0.63957456 | TRUE | TRUE |
| chr16:7105223-7105679 | 0.85544853 | 0.60530303 | 0.86576577 | 0.84809552 | 0.58024769 | TRUE | TRUE |
| chr16:7148821-7149787 | 0.85581045 | 0.47300271 | 0.83715415 | 0.80786237 | 0.38179825 | TRUE | TRUE |
| chr16:7182672-7183737 | 0.86395082 | 0.63590467 | 0.8605618 | 0.84902617 | 0.54391371 | TRUE | TRUE |
| chr10:132331053-132332170 | 0.90259827 | 0.68489712 | 0.87159244 | 0.85794379 | 0.61143605 | TRUE | TRUE |
| chr10:133181467-133182036 | 0.89655635 | 0.68720603 | 0.89846889 | 0.8483955 | 0.67002452 | TRUE | TRUE |
| chr5:3250474-3252785 | 0.70278828 | 0.4610263 | 0.70080087 | 0.66195026 | 0.4633157 | TRUE | TRUE |
| chr17:10962079-10963791 | 0.8959935 | 0.68929326 | 0.84917516 | 0.86533464 | 0.62208913 | TRUE | TRUE |
| chr17:11329981-11330264 | 0.89932997 | 0.68141345 | 0.90083931 | 0.88041547 | 0.73717949 | TRUE | TRUE |
| chr20:41311329-41312348 | 0.83589505 | 0.59011538 | 0.80155909 | 0.81287244 | 0.55228758 | TRUE | TRUE |
| chr20:41648435-41650109 | 0.85736352 | 0.58411199 | 0.83544256 | 0.81476655 | 0.49904971 | TRUE | TRUE |
| chr12:129189651-129189745 | 0.89185709 | 0.67027637 | 0.86628734 | 0.84290675 | 0.57142857 | TRUE | TRUE |
| chr12:130348817-130349864 | 0.84202549 | 0.63943853 | 0.8767957 | 0.83624853 | 0.57205589 | TRUE | TRUE |
| chr12:130406369-130407227 | 0.92617712 | 0.70912766 | 0.8834308 | 0.89678111 | 0.62440622 | TRUE | TRUE |

TABLE 4
Summarised CG DNA methylation level for Transposable Elements
(TEs) that intersect with corrected CG-DMRs in Primed iPSCs (Primed), Naive-to-
Primed iPSCs (NtoP), TNT-Primed iPSCs (TNT), and ESCs. Genomic coordinates of
each TE are listed, as well as the Family, group (class) and individual TE type.
Methylation level values are calculated as mCG/CG (range: 0 to 1) for the entire TE for
the coordinates listed, for TE's that have any overlap with corrected CG-DMRs. (Table starts on next page).

| TE | Chromosome: | | | | | | | | |
|---|---|---|---|---|---|---|---|---|---|
| number | coordinate range | Family | Class | Element | Corrected | Primed | TNT | NtoP | ESC |
| 1 | chr2:4122788-4123191 | LTR | ERVL-MaLR | MLT1B | TRUE | 0.70279044 | 0.91048387 | 0.864043256 | 0.90387142 |
| 2 | chr3:1193810-1194122 | SINE | Alu | AluSx | TRUE | 0.68602941 | 0.89076705 | 0.859742239 | 0.90279765 |
| 3 | chr3:1744136-1744667 | LTR | ERVL-MaLR | MLT1D | TRUE | 0.666237 | 0.89625167 | 0.866030184 | 0.88620552 |
| 4 | chr3:1831736-1833063 | LINE | L2 | L2a | TRUE | 0.67981277 | 0.94715897 | 0.870105656 | 0.91819114 |
| 5 | chr3:1915992-1916301 | SINE | Alu | AluJo | TRUE | 0.70157438 | 0.90606061 | 0.930829832 | 0.92632345 |
| 6 | chr3:2120682-2120851 | SINE | MIR | MIR3 | TRUE | 0.7162037 | 0.90523969 | 0.901410985 | 0.9359959 |
| 7 | chr4:64077372-64077680 | LTR | ERVL | MLT2B1 | TRUE | 0.70833333 | 0.97222222 | 0.939102564 | 0.9689011 |
| 8 | chr4:109487149-109487439 | SINE | Alu | AluSx1 | TRUE | 0.47819208 | 0.72459133 | 0.689189189 | 0.71118931 |
| 9 | chr4:126481458-126481761 | SINE | Alu | AluY | TRUE | 0.60293428 | 0.79396862 | 0.774014898 | 0.80893545 |
| 10 | chr4:126482774-126483225 | LTR | ERV1 | LTR7 | TRUE | 0.2966008 | 0.53582418 | 0.55977536 | 0.56221911 |
| 11 | chr4:126483225-126483605 | LTR | ERV1 | HERVH-int | TRUE | 0.59186791 | 0.79619753 | 0.776535803 | 0.81120204 |
| 12 | chr5:3250513-3251268 | LTR | ERV1 | MER4A | TRUE | 0.50536817 | 0.75937393 | 0.745204378 | 0.75917542 |
| 13 | chr6:32445407-32446920 | LTR | ERVK | HERVK3-int | TRUE | 0.31073941 | 0.60326087 | 0.655353282 | 0.63684901 |
| 14 | chr7:416967-417029 | SINE | MIR | MIRc | TRUE | 0.59289811 | 0.96551724 | 0.946428571 | 0.91632217 |
| 15 | chr7:68015435-68015574 | SINE | MIR | MIRb | TRUE | 0.68601736 | 0.88541667 | 0.88454072 | 0.91492568 |
| 16 | chr7:154162418-154163446 | LINE | L1 | L1PA7 | TRUE | 0.66594517 | 0.89166667 | 0.885714286 | 0.86772999 |
| 17 | chr7:157908487-157908559 | LINE | L2 | L2 | TRUE | 0.53636364 | 0.84210526 | 0.803174603 | 0.79845455 |
| 18 | chr8:138517481-138517878 | LTR | ERVL-MaLR | MLT1J2 | TRUE | 0.55152492 | 0.86190476 | 0.870397095 | 0.87722556 |
| 19 | chr8:138518126-138519195 | LTR | ERVL-MaLR | MLT1J2-int | TRUE | 0.60431574 | 0.82608696 | 0.854413083 | 0.87325974 |
| 20 | chr8:138587788-138588077 | LTR | ERVL-MaLR | MLT1I | TRUE | 0.71003788 | 0.91293393 | 0.903722142 | 0.92042125 |
| 21 | chr8:138588480-138588802 | LINE | L2 | L2c | TRUE | 0.57407872 | 0.84916027 | 0.885086517 | 0.87133042 |
| 22 | chr8:138596723-138597101 | LTR | ERVL-MaLR | MLT1C | TRUE | 0.62743563 | 0.87490119 | 0.952758881 | 0.91475111 |
| 23 | chr8:138601328-138601695 | LTR | ERVL | MLT2C2 | TRUE | 0.60713166 | 0.91040319 | 0.890738636 | 0.92774237 |
| 24 | chr8:138651519-138653527 | LINE | L1 | L1MA8 | TRUE | 0.68095656 | 0.89416376 | 0.87888796 | 0.92121565 |
| 25 | chr8:138656940-138657380 | LINE | L1 | L1M2 | TRUE | 0.52870538 | 0.83646617 | 0.850320513 | 0.8584391 |
| 26 | chr8:138684385-138686761 | LINE | L1 | L1MD1 | TRUE | 0.66028331 | 0.8993063 | 0.887017575 | 0.89820936 |
| 27 | chr8:138708526-138708933 | LINE | L1 | L1ME2 | TRUE | 0.62359672 | 0.90225564 | 0.913839804 | 0.9255812 |
| 28 | chr8:138733045-138733528 | LINE | L1 | L1MD | TRUE | 0.54192668 | 0.90070327 | 0.869394535 | 0.91141673 |
| 29 | chr8:138756930-138758590 | LINE | L1 | L1MA3 | TRUE | 0.5896509 | 0.87390531 | 0.876738346 | 0.88144097 |
| 30 | chr8:138770383-138770571 | SINE | MIR | MIR | TRUE | 0.69717262 | 0.87993421 | 0.880065359 | 0.89857143 |
| 31 | chr8:138792394-138792611 | LINE | L1 | L1MC3 | TRUE | 0.43551727 | 0.87307692 | 0.888291381 | 0.91604267 |
| 32 | chr8:138905628-138906861 | LTR | ERVL-MaLR | MLT1G1-int | TRUE | 0.65439525 | 0.87610689 | 0.905208758 | 0.92077755 |
| 33 | chr8:138974511-138974874 | LTR | ERVL-MaLR | THE1C | TRUE | 0.76368423 | 0.92201705 | 0.921538469 | 0.96496881 |
| 34 | chr8:139001299-139002011 | LTR | ERV1 | LTR30 | TRUE | 0.62448831 | 0.86941985 | 0.887588701 | 0.88987054 |
| 35 | chr8:139014091-139015768 | LINE | L1 | L1ME1 | TRUE | 0.50303059 | 0.87597984 | 0.917581983 | 0.91261094 |
| 36 | chr8:139028674-139030204 | LTR | ERVL-MaLR | THE1C-int | TRUE | 0.52199981 | 0.86601204 | 0.858880446 | 0.85228266 |
| 37 | chr8:139062329-139062508 | LINE | L2 | L2b | TRUE | 0.71660577 | 0.93371212 | 0.924342105 | 0.94192679 |
| 38 | chr8:139121589-139122131 | LTR | ERV1 | MER67C | TRUE | 0.65202833 | 0.90891841 | 0.892142441 | 0.93179162 |
| 39 | chr8:139211817-139212193 | LTR | ERVL-MaLR | THE1D | TRUE | 0.55295376 | 0.81376344 | 0.819704516 | 0.82459263 |
| 40 | chr8:139239321-139239444 | LTR | ERVL-MaLR | MLT1M | TRUE | 0.47990196 | 0.83235294 | 0.823901099 | 0.865 |
| 41 | chr8:139243278-139243563 | SINE | Alu | AluSz | TRUE | 0.53333333 | 0.93772894 | 0.863244048 | 0.90555556 |
| 42 | chr8:139448528-139448791 | LTR | ERVL? | LTR89 | TRUE | 0.75037267 | 0.92592593 | 0.926312818 | 0.96461538 |
| 43 | chr8:139453253-139453300 | LTR | ERVL-MaLR | MLT1H | TRUE | 0.72537879 | 0.92222222 | 0.982142857 | 0.95833333 |
| 44 | chr8:139530106-139530497 | LTR | ERVL | MLT2A1 | TRUE | 0.61917547 | 0.92050298 | 0.854062369 | 0.88628756 |
| 45 | chr8:140037907-140038001 | LINE | L1 | L1ME3C | TRUE | 0.49652778 | 0.96875 | 0.88277512 | 0.92005495 |
| 46 | chr9:141045125-141045432 | SINE | Alu | AluSp | TRUE | 0.29814802 | 0.67513871 | 0.625878957 | 0.67410418 |
| 47 | chr10:132754363-132754578 | LINE | L1 | L1PA5 | TRUE | 0.39893685 | 0.67780172 | 0.639524949 | 0.63481781 |
| 48 | chr12:129567275-129567569 | SINE | Alu | AluJr | TRUE | 0.6494883 | 0.86850649 | 0.853772759 | 0.8663819 |
| 49 | chr12:129627490-129627982 | LTR | ERVL | MER21A | TRUE | 0.63080808 | 0.79107143 | 0.823338331 | 0.84043706 |
| 50 | chr12:129745063-129745690 | LINE | L1 | L1PB2 | TRUE | 0.51478938 | 0.86888889 | 0.837202672 | 0.88613527 |
| 51 | chr12:129852171-129852282 | LTR | ERVL-MaLR | MLT1K | TRUE | 0.7014881 | 0.88974359 | 0.941558442 | 0.90598485 |
| 52 | chr12:129864904-129865102 | LINE | L1 | L1MD2 | TRUE | 0.513448 | 0.7637118 | 0.698241892 | 0.74635635 |
| 53 | chr12:129883890-129883934 | LTR | ERVL-MaLR | MSTD | TRUE | 0.30384615 | 0.8125 | 0.790674503 | 0.825 |
| 54 | chr12:129922847-129923323 | LINE | L1 | L1ME3 | TRUE | 0.64114056 | 0.8423913 | 0.874047781 | 0.87214747 |
| 55 | chr12:129942484-129942787 | LTR | ERVL | LTR33 | TRUE | 0.59941915 | 0.90614618 | 0.886306847 | 0.87303126 |
| 56 | chr12:130121888-130122183 | LTR | ERVL-MaLR | MSTA | TRUE | 0.65776677 | 0.93451143 | 0.902777778 | 0.93439838 |
| 57 | chr16:6475830-6475983 | LINE | L1 | L1MB2 | TRUE | 0.56125992 | 0.82235294 | 0.81547619 | 0.85863248 |
| 58 | chr16:6819457-6819577 | LINE | L1 | L1MC | TRUE | 0.61519608 | 0.88064704 | 0.933700086 | 0.91721925 |
| 59 | chr16:6965894-6966285 | LTR | ERVL | LTR47B | TRUE | 0.7056922 | 0.88518485 | 0.937015085 | 0.91240884 |
| 60 | chr16:7031112-7031488 | LINE | L1 | L1PA13 | TRUE | 0.64395769 | 0.87762238 | 0.917045455 | 0.88153846 |
| 61 | chr16:7031488-7031847 | LTR | ERVL-MaLR | THE1B | TRUE | 0.63173795 | 0.88038793 | 0.880621844 | 0.90030405 |
| 62 | chr16:7105475-7105809 | LTR | ERVL-MaLR | MLT1L | TRUE | 0.55251958 | 0.82355021 | 0.784316561 | 0.82505719 |
| 63 | chr16:81203182-81203351 | SINE | Alu | AluJb | TRUE | 0.42873024 | 0.60960591 | 0.680526412 | 0.6515368 |
| 64 | chr17:10962917-10964498 | LTR | ERVL-MaLR | MSTC-int | TRUE | 0.68703722 | 0.85227273 | 0.865084032 | 0.89014803 |
| 65 | chr17:10968266-10968714 | LTR | ERVL | ERV3-16A3_I | TRUE | 0.66553275 | 0.8612462 | 0.860881276 | 0.87673597 |
| 66 | chr17:11037834-11038575 | LTR | ERVL | LTR82A | TRUE | 0.65257749 | 0.85586735 | 0.892784178 | 0.90443743 |
| 67 | chr17:51040059-51040869 | LINE | L1 | L1P3 | TRUE | 0.62149444 | 0.80303248 | 0.821756401 | 0.82859699 |
| 68 | chr19:52988199-52988305 | LTR | ERV1 | LTR7Y | TRUE | 0.53404326 | 0.84617021 | 0.833333333 | 0.83358251 |
| 69 | chr19:52988790-52989685 | LTR | ERVK | LTR5_Hs | TRUE | 0.39189046 | 0.67859142 | 0.634209161 | 0.63056036 |
| 70 | chr20:40761003-40761599 | LINE | L1 | L1MC2 | TRUE | 0.50056654 | 0.88603989 | 0.863036283 | 0.9012021 |
| 71 | chr20:40927586-40928281 | LINE | L1 | L1MC4 | TRUE | 0.56814019 | 0.91045481 | 0.889728246 | 0.89592172 |
| 72 | chr20:41126087-41126202 | SINE | Alu | FLAM_A | TRUE | 0.47115385 | 0.81746032 | 0.802272727 | 0.82519435 |
| 73 | chr20:41385408-41385716 | SINE | Alu | AluJr4 | TRUE | 0.71428571 | 0.87593985 | 0.918406751 | 0.9147619 |
| 74 | chr20:41518423-41518913 | LINE | RTE | L4 | TRUE | 0.65393519 | 0.84545455 | 0.841718412 | 0.89118204 |
| 75 | chr20:41847664-41848209 | LTR | ERVL-MaLR | MLT1F1 | TRUE | 0.61356616 | 0.84594595 | 0.877254446 | 0.84721081 |
| 76 | chr20:41848950-41849386 | LTR | ERVL | LTR16A1 | TRUE | 0.66084985 | 0.8837146 | 0.850690687 | 0.86707159 |
| 77 | chr20:54141638-54141806 | LINE | L1 | L1MB3 | TRUE | 0.59567203 | 0.85874878 | 0.884758742 | 0.89383999 |

TABLE 5
Marker genes expressed only in either the primed
or naïve pluripotent states.

| Pluripotency state | Gene ID | Gene name |
|---|---|---|
| Primed | ENSG00000156687 | UNC5D |
| Primed | ENSG00000146938 | NLGN4X |
| Primed | ENSG00000144724 | PTPRG |
| Primed | ENSG00000248605 | AC022140.1 |
| Primed | ENSG00000172554 | SNTG2 |
| Primed | ENSG00000133424 | LARGE1 |
| Primed | ENSG00000008083 | JARID2 |
| Primed | ENSG00000162105 | SHANK2 |
| Primed | ENSG00000176406 | RIMS2 |
| Primed | ENSG00000183166 | CALN1 |
| Primed | ENSG00000184408 | KCND2 |
| Primed | ENSG00000179915 | NRXN1 |
| Primed | ENSG00000152284 | TCF7L1 |
| Primed | ENSG00000170579 | DLGAP1 |
| Primed | ENSG00000139364 | TMEM132B |
| Primed | ENSG00000261115 | TMEM178B |
| Primed | ENSG00000228222 | AC073050.1 |
| Primed | ENSG00000150907 | FOXO1 |
| Primed | ENSG00000078295 | ADCY2 |
| Primed | ENSG00000175161 | CADM2 |
| Primed | ENSG00000185274 | GALNT17 |
| Primed | ENSG00000147601 | TERF1 |
| Primed | ENSG00000151150 | ANK3 |
| Primed | ENSG00000213468 | FIRRE |
| Primed | ENSG00000231698 | AP002856.2 |
| Primed | ENSG00000254277 | AC009446.1 |
| Primed | ENSG00000237515 | SHISA9 |
| Primed | ENSG00000203279 | AL590705.1 |
| Primed | ENSG00000185261 | KIAA0825 |
| Primed | ENSG00000182050 | MGAT4C |
| Primed | ENSG00000175497 | DPP10 |
| Primed | ENSG00000237742 | AL365259.1 |
| Primed | ENSG00000106278 | PTPRZ1 |
| Primed | ENSG00000198626 | RYR2 |
| Primed | ENSG00000254934 | LINC00678 |
| Primed | ENSG00000156049 | GNA14 |
| Primed | ENSG00000134769 | DTNA |
| Primed | ENSG00000183098 | GPC6 |
| Primed | ENSG00000011201 | ANOS1 |
| Primed | ENSG00000183230 | CTNNA3 |
| Primed | ENSG00000102290 | PCDH11X |
| Primed | ENSG00000076716 | GPC4 |
| Primed | ENSG00000171724 | VAT1L |
| Primed | ENSG00000243709 | LEFTY1 |
| Primed | ENSG00000137285 | TUBB2B |
| Primed | ENSG00000174469 | CNTNAP2 |
| Primed | ENSG00000176728 | TTTY14 |
| Primed | ENSG00000176049 | JAKMIP2 |
| Primed | ENSG00000229335 | DANT1 |
| Primed | ENSG00000251680 | AC008591.1 |
| Primed | ENSG00000197462 | AC003975.1 |
| Primed | ENSG00000245573 | BDNF-AS |
| Primed | ENSG00000154277 | UCHL1 |
| Primed | ENSG00000150471 | ADGRL3 |
| Naïve | ENSG00000154645 | CHODL |
| Naïve | ENSG00000167634 | NLRP7 |
| Naïve | ENSG00000112394 | SLC16A10 |
| Naïve | ENSG00000171794 | UTF1 |
| Naïve | ENSG00000125144 | MT1G |
| Naïve | ENSG00000267383 | AC011447.3 |
| Naïve | ENSG00000203995 | ZYG11A |
| Naïve | ENSG00000078699 | CBFA2T2 |
| Naïve | ENSG00000205358 | MT1H |
| Naïve | ENSG00000232790 | LINC01162 |
| Naïve | ENSG00000227330 | AF130417.1 |
| Naïve | ENSG00000189190 | ZNF600 |
| Naïve | ENSG00000131016 | AKAP12 |
| Naïve | ENSG00000179046 | TRIML2 |
| Naïve | ENSG00000198554 | WDHD1 |
| Naïve | ENSG00000100033 | PRODH |
| Naïve | ENSG00000174718 | RESF1 |
| Naïve | ENSG00000170542 | SERPINB9 |
| Naïve | ENSG00000260364 | AC009055.1 |
| Naïve | ENSG00000022556 | NLRP2 |
| Naïve | ENSG00000204929 | AC007389.1 |
| Naïve | ENSG00000091592 | NLRP1 |
| Naïve | ENSG00000234423 | LINC01250 |
| Naïve | ENSG00000162174 | ASRGL1 |
| Naïve | ENSG00000249988 | AC092546.1 |
| Naïve | ENSG00000237764 | ENSG00000237764 |
| Naïve | ENSG00000237136 | C4orf51 |
| Naïve | ENSG00000251027 | LINC01950 |
| Naïve | ENSG00000251567 | AC018680.1 |
| Naïve | ENSG00000251574 | AC099520.1 |
| Naïve | ENSG00000234948 | LINC01524 |
| Naïve | ENSG00000254101 | LINC02055 |
| Naïve | ENSG00000249174 | AC106744.1 |
| Naïve | ENSG00000165186 | PTCHD1 |
| Naïve | ENSG00000122912 | SLC25A16 |
| Naïve | ENSG00000142182 | DNMT3L |
| Naïve | ENSG00000100181 | TPTEP1 |
| Naïve | ENSG00000153253 | SCN3A |
| Naïve | ENSG00000237534 | ENSG00000237534 |
| Naïve | ENSG00000196350 | ZNF729 |
| Naïve | ENSG00000188822 | CNR2 |
| Naïve | ENSG00000233067 | PTCHD1-AS |
| Naïve | ENSG00000105717 | PBX4 |
| Naïve | ENSG00000137948 | BRDT |
| Naïve | ENSG00000122025 | FLT3 |
| Naïve | ENSG00000163435 | ELF3 |
| Naïve | ENSG00000163286 | ALPG |

1. A method of producing an induced pluripotent stem (iPSC), the method comprising the following steps in order:
(a) culturing a somatic cell in a first culture condition adapted to promote the reprogramming of the cell towards a pluripotent state;
(b) culturing the cell in a second culture condition adapted to promote a hypomethylated DNA state, wherein the culturing in the second culture condition is for a period of time that is insufficient to allow the cell to achieve an established naïve pluripotent state; and
(c) culturing the cell in a third culture condition adapted to promote a primed pluripotent state,
thereby producing an iPSC from a somatic cell.

2. A method for reprogramming a somatic cell to a pluripotent cell, the method comprising the following steps in order:
(a) increasing the protein expression or amount of one or more factors in the somatic cell, wherein the factors are for reprogramming the somatic cell towards a pluripotent state;
(b) culturing the cell in a first culture medium, for a sufficient time and under conditions to allow the reprogramming of the cell towards a pluripotent state;
(c) culturing the cell in a second medium adapted to induce a hypomethylated DNA state, for a sufficient time and under conditions to reset the epigenetic profile of the cell; and
(d) culturing the cell in a third culture medium adapted to induce a primed pluripotent state, for a sufficient time and under conditions to convert the cell to a primed pluripotent state;
thereby reprogramming the somatic cell to a pluripotent cell.

3. A method for reprogramming a somatic cell to a pluripotent cell, the method comprising the following steps in order:
(a) increasing the protein expression or amount of one or more factors in the somatic cell, wherein the factors are for reprogramming the somatic cell towards a pluripotent state;
(b) culturing the cell in a first culture medium, for a sufficient time and under conditions to allow the reprogramming of the cell towards a pluripotent state;
(c) contacting the cell with a culture medium adapted to induce a hypomethylated DNA state and culturing the cell for a sufficient time and under conditions to allow reprogramming of the cell towards a hypomethylated DNA state;
(d) contacting the cell with a primed culture medium and culturing the cell for a sufficient time and under conditions to allow reprogramming of the cell to a primed pluripotent state;
thereby reprogramming the somatic cell to a pluripotent cell.

4. The method of any one of claims 1 to 3 wherein the iPSC has an epigenomic profile that is at least 75% similar, 80% similar, 90% similar or greater, to the epigenomic profile of an embryonic stem cell, optionally wherein the epigenomic profile is determined by reference to non-CG methylation levels.

5. The method of any one of claims 1 to 4, wherein the somatic cell is a human somatic cell and the iPSC produced according to the method is a human iPSC.

6. The method of any one of claims 1 to 5, wherein, the time for culturing the cell to commence reprogramming towards a pluripotent state is at least 1 day following contacting the somatic cell with the first culture medium or following increasing the protein expression, or amount of the one or more factors.

7. The method of claim 6, wherein the time is 2, 3, 4, 5, 6, 7, or more days after contacting the cell with the first culture medium, or increasing the protein expression, or amount of the one or more factors.

8. The method of any one of claims 1 to 7, wherein the time for culturing the cell to commence reprogramming towards a pluripotent state may be any period of time that promotes the reduction of markers and characteristics associated with the somatic cell.

9. The method of any one of claims 1 to 8, wherein the second culture condition or second culture medium is any culture condition or medium adapted for promoting a naïve pluripotent state.

10. The method of claim 9, wherein the medium comprises a MEK inhibitor, a PKC inhibitor, a GSK3 inhibitor, a STAT3 activator, human Leukaemia inhibitor factor (hLIF) and a ROCK inhibitor.

11. The method of claim 9, wherein the medium is selected from the group consisting of: T2iLGoY, 5iLAF, RSeT, PXGL and NHSM as herein described.

12. The method of any one of claims 1 to 11, wherein the third culture condition or third culture medium comprises any culture medium adapted for promoting a primed pluripotent state.

13. The method of claim 12, wherein the medium is selected from the group consisting of: Essential 8, KSR/FGF2, mTeSR, AKIT or B8 as herein described.

14. The method of any one of claims 1 to 13, wherein the culturing of the cell in the second culture condition or second culture medium is for a period of time that is 21 days or less.

15. The method of claim 14, wherein the period of time is 13 days or less, optionally 10 days or less, or 5 days or less.

16. The method of any one of claims 2 to 15, wherein culturing the cell in the second culture medium is performed for a period of time and in conditions that are not sufficient to achieve a naïve pluripotent state for the cell and wherein step d) is performed before the cell has completed reprogramming and before the cell achieves a naïve pluripotent state.

17. The method of any one of claims 2 to 15, wherein culturing the cell in the second culture medium is performed for a period of time and in conditions that achieve a naïve pluripotent state for the cell and wherein step d) is performed after the cell has achieved a naïve pluripotent state.

18. The method of any one of claims 1 to 17, wherein a naïve pluripotent state comprises a cell phenotype that includes cells that are round, dome-shaped.

19. The method of any one of claims 1 to 18, wherein a primed pluripotent state comprises a cell phenotype characterised by the presence of flat cell colonies.

20. The method of any one of claims 1 to 19, wherein a naïve pluripotent state further comprises the expression of 1, 2, 3, 4, 5, 6, 7, 8, 9, 10, 11, 12, 13, 14, 15, 16, 17, 18, 19, 20, 21, 22, 23 or all markers selected from: KLF2, KLF4, TFCP2L1, TBX3, REX1, GBX2, STELLA (DPPA3), KLF17, DPPA5, TFCP2L1, MAEL, UTF1, ZFP57, DNMT3L, FGF4, FOXR1, ARGFX, TRIM60, DDX43, BRDT, ALPPL2, KHDC3L, KHDC1L and PRAP1.

21. The method of any one of claims 1 to 20, wherein a primed pluripotent state further comprises the expression of 1, 2, 3, 4, 5, 6, 7, 8, 9, 10, 11, 12, 13, 14, 15 or all of markers selected from: SFP, EOMES, BRACHYURY, OTX2, ZIC2, ZIC3, ZIC5, DNMT3B, KDR, CDH2, CER1, COL2A1, DAZL, TCF7L1, SOX11 and SALL2.

22. The method of any one of claims 1 to 14, or 16 to 21, wherein the cell is cultured in the second culture conditions or second culture medium for a period of 0.5 days, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days prior to being contacted with the third culture medium or third culture conditions.

23. The method of any one of claims 1 to 22, wherein the cell is cultured in the third culture medium or third culture condition for at least about 0.5 days, 1 day, 2 days, 3 days, 4 days, 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days or about 21 days.

24. The method of any one of claims 1 to 23, wherein step a) comprises contacting the cell with an agent for increasing the expression or amount of one or more of the factors for reprogramming the somatic cell towards a pluripotent state.

25. The method of claim 24, wherein the agent is selected from the group consisting of: a nucleotide sequence, a protein, an aptamer and small molecule, ribosome, RNAi agent and peptide-nucleic acid (PNA) and analogues or variants thereof.

26. The method of claim 25, wherein the agent is a nucleic acid and wherein the nucleic acid is provided for contact with the cell in the form of a vector, preferably wherein the vector is a viral vector.

27. The method of any of claims 24 to 26, wherein the factors are selected from the transcription factors: OCT4, SOX2, KLF4 and MYC of variants thereof.

28. The method of any one of claims 24 to 27, wherein the factors comprise all four of the transcription factors OCT4, SOX2, KLF4 and MYC (OSKM), or variants thereof.

29. The method of claim 27 or 28, wherein the transcription factors further comprise the factors LIN28 and/or NANOG.

30. An induced pluripotent stem cell (iPSC) or isolated iPSC obtained or obtainable by any one of claims 1 to 29.

31. A population of cells comprising an induced pluripotent stem cell (iPSC), obtained or obtainable according to the method of any one of claims 1 to 29, preferably wherein at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population are iPSCs and those iPSCs obtained or obtainable according to the method of any one of claims 1 to 29.

32. A differentiated cell or isolated differentiated cell, primordial germ cell or gamete derived from an iPSC obtained or obtainable according to the method of any one of claims 1 to 29.

33. A population of differentiated cells, primordial germ cells or gametes derived from an iPSC obtained or obtainable according to the method of any one of claims 1 to 29, wherein preferably at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100% of the cells in the population are differentiated cells and those differentiated cells are derived from iPSCs obtained or obtainable according to the method of any one of claims 1 to 29.

34. An organoid or organised collection of cells derived from a population of iPSCs or population differentiated cells of claim 32 or 33.

35. A pharmaceutical composition comprising:
an iPSC or isolated iPSC of claim 30;
a population of iPSCs of claim 31;
a differentiated cell or an isolated differentiated cell of claim 32;
a population of differentiated cells of claim 33; or
an organoid or organised collection of cells of claim 34 or part thereof;
and a pharmaceutically acceptable excipient.

36. A method of treating a disease or condition requiring administration of an iPSC or population of cells, comprising administering to a subject in need thereof:
an iPSC or isolated iPSC of claim 30;
a population of iPSCs of claim 31;
a differentiated cell or an isolated differentiated cell of claim 32;
a population of differentiated cells of claim 33;
an organoid or organised collection of cells of claim 34 or part thereof; or
a pharmaceutical composition of claim 35.

37. An iPSC or isolated iPSC of claim 30, a population of iPSCs of claim 31, a differentiated cell or an isolated differentiated cell of claim 32, a population of differentiated cells of claim 33, an organoid or organised collection of cells of claim 34 or part thereof; or a pharmaceutical composition of claim 35, for use in treating a disease or condition requiring administration of an iPSC or population of cells.

38. A kit for producing an iPSC according to the method of any one of claims 1 to 29, optionally wherein the kit comprises reagents for culturing a somatic cell and written instructions for performing the method of any one of claims 1 to 29.
